(19) United States
(12) Patent Application Publication
(10) Pub. No.: US 2024/0409647 A1
(43) Pub. Date: Dec. 12, 2024

(54) BISPECIFIC BINDING PROTEINS AGAINST ALARMINS AND USES THEREOF

(71) Applicant: SINOMAB BIOSCIENCE LIMITED, Pak Shek Kok (NT), Hong Kong (CN)

(72) Inventors: Nan SONG, Pak Shek Kok (NT), Hong Kong (CN); Lik Hang LAM, Pak Shek Kok (NT), Hong Kong (CN); Chin Wai HUI, Pak Shek Kok (NT), Hong Kong (CN); Weimin LI, Pak Shek Kok (NT), Hong Kong (CN); Shui On LEUNG, Pak Shek Kok (NT), Hong Kong (CN)

(73) Assignee: SINOMAB BIOSCIENCE LIMITED, Pak Shek Kok (NT), Hong Kong (CN)

(21) Appl. No.: 18/814,167

(22) Filed: Aug. 23, 2024

Related U.S. Application Data
(63) Continuation-in-part of application No. PCT/CN2023/077870, filed on Feb. 23, 2023.
(60) Provisional application No. 63/313,483, filed on Feb. 24, 2022.

(51) Int. Cl. C07K 16/28; A61K 45/06; A61P 37/08; C07K 16/24

(52) U.S. Cl. CPC C07K 16/2866; A61K 45/06; A61P 37/08; C07K 16/244; C07K 2317/31; C07K 2317/565; C07K 2317/622; C07K 2317/76; C07K 2317/92

(57) ABSTRACT

Provided herein are methods of treating allergic diseases, such as moderate-to-severe asthma and atopic dermatitis, methods of reducing daily dosage of oral-corticosteroid use, methods of suppressing the proliferation of innate lymphoid cells, methods of compromising the Type 1 and Type 2 immune responses with certain bispecific binding proteins (bsBp) capable of neutralizing 2 different alarmins. Exemplary bsBps, characteristics thereof, and methods of screening for additional therapeutic bsBps are also described herein.

### 1. FIELD OF THE INVENTION

The present invention relates to molecular biology and allergic diseases, specifically, to the identification and uses of bispecific antibodies comprising an alarmin receptor binding immunoglobulin G antibody (IgG) and an alarmin binding protein for the treatment of various allergic diseases, such as moderate-to-severe asthma and atopic dermatitis.

### 2. PRIORITY

This application is a continuation-in-part of International Application No. PCT/CN2023/077870 filed Feb. 23, 2023 and designating the United States, which, in turn, claims the benefit of U.S. Provisional Application No. 63/313,483 filed Feb. 24, 2022. The entire contents of both priority applications are incorporated by reference herein.

### 3. SEQUENCE LISTING

The instant application contains a Sequence Listing compliant with WIPO Standard ST.26 entitled “SBL009PCTSL_V2.xml” created Aug. 21, 2024 that is 91,621 bytes in size and hereby incorporated by reference in its entirety.

### 4. BACKGROUND OF THE INVENTION

Alarmins are endogenous, constitutively expressed, chemotactic and immune activating proteins/peptides that are released as a result of degranulation, cell injury or death or in response to immune induction.

In particular, the “alarmins” thymic stromal lymphopoietin (TSLP), Interleukin-33 (IL-33) and Interleukin-25 (IL-25) are released from barrier tissues (e.g., airway epithelium) in response to allergens. These alarmins serve as the upstream elements responsible for initiating Th2 immune responses via the activation of type 2 innate lymphoid cells (ILC2s) and Th2 cells, leading to a cascade of events such as the release of IL-4, IL-5, IL-13 and IgE, and resulting in the manifestation of a multitude of allergic responses. Biologics targeting any one of these alarmins and cytokines produce variable improvements in the symptom scores of allergic responses such as asthma and atopic dermatitis. Although single use of anti-TSLP or anti-IL-33 antibodies has been clinically validated in treating asthma, the lack of complete efficacy, however, may be due to the fact that each therapy targets only some of the elements of the pathways that regulate type 2 inflammation, leaving other elements of the disease pathophysiology unattended. These alarmins should potentially interact with one another contributing to the respective inflammatory responses. Using chronic models of helminth infection and type 2 cytokine-driven lung inflammation, it was demonstrated that targeting all three alarmins (TSLP, IL-25 and IL-33) appeared to be more efficacious than blocking any one single alarmin alone [Vannella, Kevin M et al. Science Translational Medicine vol. 8,337 (2016): 337ra65.]. Furthermore, disruption of all three mediators in a model of chronic house dust mite-induced allergic lung inflammation resulted in reduced inflammation, mucus production and lung remodeling, suggesting these alarmins interact with each other to potentiate their individual effects in the maintenance of type 2 pathology. Hence, there is a great need novel clinical methods in which two or more of these disease-causing alarmins are suppressed to thereby improve clinical outcomes against allergic diseases, such as the reduction of steroid-use and maintenance of long-term disease remission. Methods provided in the present disclosure meet this need and provide the related advantages in the attenuation of disease progression.

### 5. SUMMARY OF THE INVENTION

Bearing in mind the clear need in the art for new methods of treating allergic diseases and disorders, it is an objective of the present invention to provide bispecific antibodies (bsBps) or antibody-receptor fusion proteins against two different alarmins characterized by one or more of the following activities: i) binding to purified human IL-17RB and IL-33 proteins with the K_{D} lower than 10⁻⁸M; ii) binding to purified human IL-17RB and TSLP proteins with the K_{D} lower than 10⁻⁸M; iii) inhibiting the releases of Th2 related cytokines to a greater extent compared to anti-IL17RB monoclonal antibody; iv) inhibiting the proliferation and activation of ILC2s to a greater extent compared to anti-IL17RB monoclonal antibody.

As demonstrated herein, such bispecific antibodies (bsBps) or antibody-receptor fusion proteins have the potential to prevent, suppress or/and delay the progression of allergic diseases, such as asthma and atopic dermatitis (AD). Accordingly, it is an objective of the present invention to provide a method of treating a disease or disorder related to allergy in a subject in need thereof that includes the step of administering to the subject a therapeutically effective amount of a bispecific binding protein against 2 different alarmins X and Y, wherein the bispecific binding protein is composed of (a) anti-alarmin X receptor IgG and (b) anti-alarmin Y scFv and (c) polypeptide linker.

In a preferred embodiment, the subject is a human afflicted with clinical or pre-clinical asthma, atopic dermatitis, fibrotic disease, inflammatory bowel disease (IBD), Crohn's disease, ulcerative colitis, chronic obstructive pulmonary disease, chronic sinusitis, chronic rhinosinusitis with nasal polyps.

In particularly preferred embodiments, the alarmin X receptor is IL-17RB, the alarmin Y receptor TSLP or IL-33, and the alarmin receptor is ST2 or TSLPR.

In other preferred embodiments, the anti-alarmin X IgG is selected from among an IgG1 antibody, an IgG2 antibody, an IgG3 antibody, and an IgG4 antibody.

It is another objective of the present invention to provide a method of treating a disease or disorder related to allergy in a subject in need thereof that includes the step of administering to the subject a therapeutically effective amount of bispecific binding protein capable of (a) blocking IL-25 and IL-33 signaling and/or (b) blocking IL-25 and TSLP signaling.

In a preferred embodiment, the subject, preferably a human, is afflicted with clinical or pre-clinical asthma, atopic dermatitis, fibrotic disease, inflammatory bowel disease (IBD), Crohn's disease, ulcerative colitis, chronic obstructive pulmonary disease, chronic sinusitis, chronic rhinosinusitis with nasal polyps.

In particularly preferred embodiments, the bispecific binding protein is selected from among an anti-IL-17RB/anti-human TSLP bispecific antibody, an anti-IL-17RB/anti-human IL-33 bispecific antibody, an anti-IL-17RB/human ST2 antibody-receptor fusion protein, and an anti-IL-17RB/human TSLPR antibody-receptor fusion protein.

In other preferred embodiments, the bispecific binding protein includes a light chain variable region (VL) composed of a VL CDR1, VL CDR2, and VL CDR3 that have the amino acid sequences of SEQ ID NO: 4, SEQ ID NO: 5, and SEQ ID NO: 6, respectively, and a heavy chain variable region (VH) composed of a VH CDR1, VH CDR2, and VH CDR3 that have the amino acid sequence of SEQ ID NO:1, SEQ ID NO:2, and SEQ ID NO:3, respectively.

In particularly preferred embodiment, the VL and VH of the anti-IL-17RB antibody have the amino acid sequences of SEQ ID NO:33 and SEQ ID NO:32, respectively, and/or a light chain sequence of SEQ ID NO: 33 and a heavy chain sequence of any one selected from SEQ ID NOs: 60-74.

In preferred embodiments, the bispecific antibodies (bsBps) and/or antibody-receptor fusion proteins are administered intravenously, intramuscularly, subcutaneously, intracranially, intrathecally, intraventricularly, intraperitoneally, intranasally, parenterally, topically, or intradermally, optionally in conjunction with a second therapeutic agent, examples of which include, but are not limited to a corticosteroid, a DNA methyltransferase (DNMT) inhibitor, an anti-IL17A antibody, an anti-IL12/IL23 antibody, an anti-IL23 antibody, an anti-IL17RA antibody, and a tyrosine kinase inhibitors.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims.

### 6. BRIEF DESCRIPTION OF DRAWINGS

FIG. 1 depicts the dose dependent curve of SM17 binding to IL-17RB from different species.

FIG. 2 depicts the binding specificity of SM17 to IL-17 receptor family members.

FIG. 3 depicts SM17 binding to native human Il-17RB.

FIG. 4: depicts the dose dependent inhibition of IL-5 by SM17 on human PBMC cultures.

FIG. 5 depicts IL-8 production from TK-10 cells was inhibited by SM17.

FIG. 6 depicts the effect of SM-17 in suppressing airway hyperresponsiveness of OVA-induced murine experimental asthma.

FIG. 7 depicts the effect of SM-17 in suppressing BALF IL-5 and IL-13 levels in OVA-induced murine experimental asthma.

FIG. 8 depicts the effect of SM-17 in suppressing BALF eosinophil cell counts in OVA-induced murine experimental asthma.

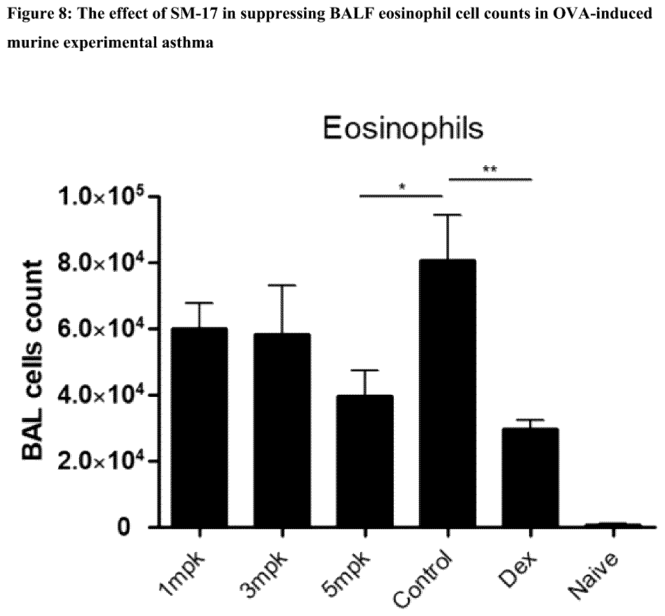

FIG. 9 depicts modes of IL25/SM17 interactions and possible disease indications.

FIG. 10A depicts the design of bispecific binding proteins (bispecific antibody and antibody-receptor fusion protein).

FIG. 10B depicts the SDS-PAGE of SM17, SM17-anti-TSLP, and SM17-anti-IL-33 bsBp purified from ExpiCHO transient transfection system.

FIG. 11 depicts the antigen binding of anti-alarmin bispecific binding proteins

FIG. 12 depicts the induction of IFNγ, CCL8, CCL17, IL-5 and IL-13 from human PBMC by alarmins and the inhibitory effects of SM17 on cytokine release.

FIG. 13 depicts the potencies of different bispecific binding proteins on cytokine and chemotactic factor releases from induced human PBMC.

FIG. 14 depicts the results of pro-proliferative effects of alarmins on ILC2s.

FIG. 15 depicts the results of the response of ILC2s and Th2 cells to steroid hormone and bsBp.

FIG. 16 depicts the results of dendritic cell potency assay.

### 7. DETAILED DESCRIPTION OF THE PREFERRED EMBODIMENTS

Type II inflammatory diseases involve a plethora of stimulatory alarmins and cytokines upon challenges with allergens. Atopic dermatitis (AD) and asthma are the representative diseases of this class.

AD is a chronic, inflammatory skin disease characterized by severe itchiness. It affects 15-30% of children and 2-10% of adults with seriously compromised quality of life. Immunological factors of AD pathogenesis include numerous disorders of Th2 lymphocytes and the release of associated cytokines, such as IL-4, IL-5 and IL-13. These factors lead to elevated production of IgE, resulting in increased inflammation in the skin, and aggravating the skin barrier defect in patients with AD.

Of particular interests are interleukin-4 (IL-4) and Interleukin-13 (IL-13): they are the signature cytokines of the type II inflammatory response for AD, triggered either by an invading parasite or allergen. The receptors for IL-4 and IL-13 share a common receptor chain, namely, IL-4Rα; IL-4Rα/IL-2Rγc (γc) heterodimer constitutes the receptor for IL-4, whereas IL-4Rα/IL-13Rα1 heterodimer constitutes the receptor for IL-13. Binding of the IL-4 or IL-13 to the IL-4Ra receptor chain will allow further association of the IL-4/IL-4Rα complex to the γc receptor chain, or IL-13/IL-4Rα complex to the IL-13Rα1 receptor chain, respectively. Although the receptor chain IL-4Rα is widely expressed, albeit at low levels in some cell types, expression of γc or IL-13Rα1 receptor chains are cell type restricted. For example, in non-hematopoietic cells, while IL-13Rα1 demonstrates somewhat higher expression, γc expression is either low or absent [Junttila, Ilkka S et al. The Journal of Experimental Medicine, vol. 205, 11 (2008): 2595-608.]. Recent studies depicted a central role played by IL-4 towards T2 responses within the lymph nodes in generating and regulating humoral immunity involving IgE; whereas IL-13 played a more prominent role in the peripheral tissues. Both IL-4 and IL-13 significantly decrease the expression of key structural proteins like filaggrin, filaggrin 2, loricrin, involucrin, keratin 1, keratin 10, hornerin, desmoglein, and desmocollin 1, as well as the lipid composition important for normal skin barrier function, leading to increased transepidermal water loss (TEWL) typically measured to reflect the severity and even used to predict the occurrence of AD. Additionally, by suppressing AMP production in keratinocytes, both IL-4 and IL-13 were reported to be responsible for the development of dysbiosis of the skin, typically characterized by a strong colonization with Staphylococcus aureus; the occurrence of which has recently been shown to precede the appearance of AD lesions. Towards these ends, 2 biologics addressing these pathways were developed and approved for the treatment of moderate-to-severe AD. They are namely, Dupilumab (anti-IL-4Ra antibody inhibiting both IL-4 and IL-13 responses) and Tralokinumab (anti-IL-13 antibody). However, about 50% of AD patients become unresponsive to these antibodies 1-year post-treatment. [Bangert, Christine et al. Science Immunology, vol. 6, 55 (2021): eabe2749; Wollenberg, A et al., The British Journal of Dermatology vol. 184, 3 (2021): 437-449.]

Asthma is a chronic inflammatory disorder of the airways characterized by bronchial hyperresponsiveness and variable airflow limitation. Asthma affects more than 300 million people worldwide [Braman, Sidney S. Chest, vol. 130, 1 Suppl (2006): 4S-12S.]. Although the majority of patients with asthma can achieve disease control with standard controller therapy, approximately 5-10% have severe asthma that remains inadequately controlled despite adherence to standard treatment (the high-dose inhaled corticosteroid (ICS) plus long-acting beta-agonists (LABA)). For those severe asthma uncontrolled by standard treatment, the Global Initiative for Asthma (GINA) guidelines recommend the use of oral corticosteroids (OCS) for maintenance therapy. However, OCS-related adverse events, such as those affecting the cardiovascular, gastrointestinal, and musculoskeletal systems, as well as infections, are common and can sometimes be fatal. Hence, severe asthmatic patients are characterized as having the most urgent unmet medical needs and can be eligible to add-on biological therapies.

There are 2 major categories of asthma: Th2 high and Th2 low. In Th2 high asthma, exaggerated production of type 2 immune cytokines such as IL-4, IL-5, and IL-13 can lead to pulmonary eosinophilia, elevated immunoglobulin (Ig) E-levels, increased mucus production, and life-threatening problems in breathing [Kuruvilla, Merin E et al., Clinical Reviews in Allergy & Immunology, vol. 56, 2 (2019): 219-233]. Therefore, biologics targeting IgE, IL-4, IL-5 and IL-13 have recently emerged as a promising add-on therapy for severe uncontrolled asthma with Th2 high phenotypes.

IL-4 and IL-13 are also thought to have some nonredundant functions in allergy and asthma. In particular, IL-4 is considered to act predominantly in the early phase of asthma development through its role in regulating T cell proliferation and survival, and IgE synthesis. In contrast, due to the lack of surface IL13Ra1 expression, human T cells could not respond to IL-13. Unlike IL-4, IL-13 is more predominantly involved in late phases of allergic reactions, such as airway remodeling and mucus hypersecretion by goblet cells, fibrosis, smooth muscle alterations, and increased airway hyperreactivity [Gour, N., & Wills-Karp, M. (2015). Cytokine, 75(1), 68-78].

However, the clinical outcomes for antibodies targeting IL-4 (altrakincept, pascolizumab) or IL-13 (tralokinumab) alone for the treatment of asthma were disappointing [Panettieri, Reynold A Jr et al. The Lancet. Respiratory Medicine vol. 6, 7 (2018): 511-525; Bagnasco, Diego et al. International Archives of Allergy and Immunology, vol. 170, 2 (2016): 122-31.]. But when both IL-4 and IL-13 were targeted, as in the case of Dupilumab, effective treatment of Th2 mediated asthma was clinically demonstrated [Maspero, Jorge F et al. The Journal Of Allergy And Clinical Immunology. In practice, vol. 8, 2 (2020): 527-539.e9.]. This result suggests that dual blockade of IL-4 and IL-13 is necessary in the treatment of Th2 mediated asthma.

IL-5 exerts a central pathogenic role in the differentiation, recruitment, survival, and degranulation of eosinophils [Pelaia, Corrado et al. Frontiers in Physiology, vol. 10 1514. 17 Dec. 2019]. There are a significant number of patients with severe asthma that express a Th2-high phenotype featured by eosinophilic inflammation. Airway eosinophilia can occur in more than half of the asthmatic subjects, and high eosinophil levels are associated with recurrent asthma exacerbations and severe bronchial obstruction. Th2-high asthma with eosinophilia is often therapeutically responsive to corticosteroids, probably via the removal of eosinophil by corticosteroid induced apoptosis. However, severe eosinophilic asthma may be resistant to both inhaled and systemic corticosteroids due to an excessive bronchial amount of IL-5. The excessive bronchial amount of IL-5 can overcome the pro-apoptotic effects of corticosteroids on eosinophils. Blocking IL-5 activities by anti-IL-5 antibodies can therefore help to sustain the therapeutic responsiveness of corticosteroid treatment, and in fact 3 antibodies blocking IL-5 pathway are being used in conjunction with corticosteroid for the treatment of Th2 high asthma; including Reslizumab (anti-IL-5 antibody), Mepolizumab (anti-IL-5 antibody) and Benralizumab (anti-IL-5Ra antibody).

Allergic asthma, a subtype of Th2-high asthma, is characterized by the presence of IgE antibodies against one or more common environmental allergens, such as house dust mite. In patients with allergic asthma, anti-allergen IgE binds to IgE receptor (FcεRI) on the surface of mast cell. Exposure to allergen antigen can lead to crosslinking of mast cell surface FcεRI; mast cells are activated when such FcεRI crosslinking is of sufficient strength and duration, resulting in the release of the autacoid mediators: histamine, prostaglandin (PG) D2, and leukotriene (LT) C4, finally leading to bronchoconstriction, mucus secretion, and mucosal edema. Activated mast cells can also synthesize and secrete a large number of proinflammatory cytokines (including IL-4, IL-5, and IL-13), which can in turn regulate both IgE synthesis and the development of eosinophilic inflammation. Omalizumab, an anti-IgE antibody, was the first, and for a long time the only available monoclonal antibody for add-on treatment of severe allergic asthma. Omalizumab functions by selectively preventing human IgE from binding to its receptors and therefore suppressing mast cell activation.

Although much progress has been made in elucidating Th2-high inflammation pathways and the development of relevant biologics for treating Th2-high asthma, effective approach addressing Th2-low asthma is still lacking. The problem is further aggravated by the fact that Th2-low asthmatic patients respond poorly to corticosteroids. Thus, there is an unmet medical need for treatment modalities for the effective treatment of Th2-low asthma.

Another problem in asthma standard care is the over-reliance on OCS. Around 30% of adult patients with severe asthma rely on OCS therapy in addition to ICS in order to maintain an acceptable level of asthma control [Chung, Kian Fan et al. The European Respiratory Journal, vol. 43, 2 (2014): 343-73.]. The Global Initiative for Asthma (GINA) group committed to setting an “acceptable” OCS dose for maintenance therapy and they suggested 7.5 mg daily as the acceptable level as it corresponds to the physiological level of steroid production. However, recent research has revealed that there is no benign OCS prescription, as the risk of adverse events is cumulative and escalates with each treatment. An increase in morbidity is seen after only four short courses of OCS [Sullivan, Patrick W et al. The Journal of Allergy and Clinical Immunology, vol. 141, 1 (2018): 110-116.e7.]. In terms of lifetime cumulative doses, a majority of the side effects emerged after patients were treated with 1-2.5 g of corticosteroid, and the incidence of diabetes started to increase in patients receiving only 0.5 g of corticosteroid [Price, David B et al. Journal of Asthma and Allergy, vol. 11 193-204. 29 Aug. 2018,].

The OCS-sparing effects of dupilumab, mepolizumab, reslizumab, omalizumab, and benralizumab were clinically evaluated. Although treatment with these biologics could reduce 50% daily OCS doses, only 5%-20% severe asthmatic patients were able to discontinue OCS usage. There remains a huge unmet medical need to develop new treatment modalities or biologics for treating asthma that could eliminate or further mitigate the reliance on OCS [Cataldo, Didier et al. The Journal of Asthma: Official Journal Of The Association For The Care Of Asthma, vol. 58, 4 (2021): 448-458.].

Current FDA-approved biologics for treating asthma and AD all target downstream pathways of Th2 inflammation. There are, however, clinical inadequacies that need to be addressed in patients treated with these biologics, such as the high rate of relapse, limited efficacy on non-Th2 inflammation, and reliance on continued OCS use, albeit at reduced levels. Recently, new biologics are being developed to target alarmins, including IL-25, TSLP, and IL-33. They are the upstream elements affecting Th2 inflammatory pathways and could potentially contribute to both Th2 and non-Th2 inflammatory responses. Therefore, anti-alarmin biologics may provide additional options that could address the current clinical inadequacies for patients with AD and Th2-high/low severe asthma.

IL-25 (also known as IL-17E) is a member of the IL-17 cytokine family that covers IL-17A to IL-17F. IL-25 binds to its receptor composed of IL-17 receptor A (IL-17RA) and IL-17 receptor B (IL-17RB) for signal transduction [Borowczyk, Julia et al. The Journal Of Allergy And Clinical Immunology, vol. 148, 1 (2021): 40-52.]. IL-25 is a type 2 cytokine produced by Th2 cells, and is capable of inducing IL-4, IL-5 and IL-13 gene expression and further amplifying allergic inflammatory response in the lung and the digestive tract. IL-25 is important in type 2 immune response because it activates the IL-17RA/IL-17RB complex in a variety of cell types, including epithelial cells, Th2 cells and ILC2s. IL-25 is reported to inhibit CD4⁺ T-cell activation and differentiation into Th17 cells and play an anti-inflammatory role in autoimmune and inflammatory diseases through the downregulation of Th1 and Th17 cell responses.

Thymic stromal lymphopoietin (TSLP) is a member of the IL-2 cytokine family, and a distant paralog of IL-7. TSLP binds to a heterodimeric receptor formed by a TSLP-specific TSLPR subunit and the IL-7R signaling chain to act on several immune cell types including dendritic cells, ILC2s, mast cells, basophils, and T cells. During allergic inflammation, the primary producers of TSLP are epithelial cells, keratinocytes and stromal cells. TSLP has a critical role in driving Th2-mediated inflammation by modulating antigen-presenting cells (e.g., Dendritic cells) to amplify Type 2 cytokines by T cells and innate lymphoid cells [Ito, Tomoki et al. The Journal Of Experimental Medicine, vol. 202, 9 (2005): 1213-23].

IL-33 is a member of the IL-1 family and was recently identified as the ligand for T1/ST2 (ST2), a member of the IL-1 receptor family. IL-33 is a dual function protein acting both as a proinflammatory cytokine and an intracellular nuclear factor with transcriptional regulatory properties. After cell stress or necrosis, IL-33 is released into the extracellular space and functions as an endogenous danger signal that alerts the immune system of tissue damage during trauma or infection. IL-33 amplifies both Th1- and Th2-type responses through its activity on human basophils, allergen-reactive Th2 cells, iNKT and NK cells. While IL-33 canonically triggers type 2 cytokine responses, this cytokine can also synergize with type 1 cytokines like IL-12 to provoke interferon-gamma (IFNγ) [Komai-Koma, Mousa et al. Immunobiology, vol. 221, 3 (2016): 412-7]. IL-33 is thus emerging as a crucial immune modulator with important roles in allergic, fibrotic, infectious, and chronic inflammatory diseases.

As IL-25, TSLP, and IL-33 (collectively known as alarmins) exhibit broad functions beyond Th2 immune response, biologics against these alarmins are believed to be effective in additional allergic disease subtypes other than indications that were approved for biologics interacting with targets that are downstream of the Th2 pathway. Both anti-TSLP and anti-IL33 antibodies have been clinically evaluated in asthma and AD [Simpson, Eric L et al. Journal of the American Academy of Dermatology, vol. 80, 4 (2019): 1013-1021; Chen, Yi-Ling et al. Science Translational Medicine, vol. 11,515 (2019): eaax2945]. Tezepelumab (AMG 157/MEDI9929) is a fully human immunoglobulin G2-lambda monoclonal antibody that binds specifically to TSLP and prevents TSLP from interacting with its receptor complex. In a Phase II, randomized, double-blind, placebo-controlled study (ALLEVIAD; NCT02525094), patients with moderate-to-severe AD demonstrated a trend towards improvements for all endpoints upon treatment with Tezepelumab plus topic use of corticosteroid (TCS) over placebo plus TCS, however, such improvements failed to achieve statistical significance, as assessed by EASI50 at week 12. Two separate Phase III trials (SOURCE and NAVIGATOR) were performed to evaluate the clinical efficacies of Tezepelumab for treating moderate-to-severe asthma. In the NAVIGATOR study, patients with moderate-to-severe asthma were treated either with Tezepelumab plus OCS or with placebo plus OCS. Primary endpoints were met in the treatment group compared to that of the placebo group with statistical significance, demonstrating a clinically meaningful reduction in AAER (Annual Asthma Exacerbation Rate) [Menzies-Gow, Andrew et al. The New England Journal of Medicine, vol. 384, 19 (2021): 1800-1809]. In the NAVIGATOR study, Tezepelumab (used concomitantly with OCS) are equally effective to suppress Th2-high and Th2-low severe asthma. However, in the SOURCE study, Tezepelumab failed to meet the primary endpoint, i.e., reduction in the daily OCS without loss of asthma control, compared to placebo with statistical significance. This result suggested that Tezepelumab used as monotherapy may not be sufficient for treating moderate-to-severe asthma.

Etokimab (ABN020), an anti-IL-33 humanized IgG1 monoclonal antibody, were clinically evaluated for the treatment of AD. In a Phase 1 trial, administration of ABN020 resulted in a strong reduction of blood eosinophils count. ABN020 was found to significantly alleviate the symptoms of home dust mite-induced dermatitis and reduce neutrophils skin infiltration (a hallmark of non-Th2 inflammation) in a Phase Iia study [Chen, Yi-Ling et al. Science Translational Medicine, vol. 11,515 (2019): eaax2945]. These results suggested that ABN020 could suppress both Th2 and non-Th2 inflammation. However, ABN020 failed to meet its primary end point for the treatment of moderate-to-severe AD in a subsequent Phase lib study. Another anti-IL-33 antibody, Itepekimab (REGN3500), was clinically evaluated for the treatment of asthma. In a Phase II proof-of-concept (POC) study, patients who discontinued the use of ICS and long-acting bronchodilator inhalers (LABA) received treatments with either dupilumab or itepekimab for 12 weeks [Wechsler, Michael E et al. The New England Journal Of Medicine, vol. 385, 18 (2021): 1656-1668.]. The rate of loss of asthma control in patients treated with itepekimab (22%) is significantly lower than that receiving placebo (41%). This data suggested that itepekimab may be effective as monotherapy without ICS and LABA. However, the efficacy of Itepekimab was slightly inferior to that of the anti-IL4Ra antibody Dupilumab (19%). Further clinical development of Itepekimab on asthma was therefore discontinued from strategical rather than scientific considerations. Regardless, itepekimab was later found to reduce the exacerbation rate and improve lung function in former smokers with chronic obstructive pulmonary disease (COPD), a disease characterized by neutrophilic inflammation [Rabe, Klaus F et al. The Lancet. Respiratory Medicine, vol. 9, 11 (2021): 1288-1298]. Two Phase III clinical studies with itepekimab for the treatment of COPD in former smokers are ongoing.

So far, the efficacies of intercepting the IL-25/IL-17RB pathway on immunological diseases such as asthma and AD have not been clinically validated. The most advanced anti-IL25 antibody is XKH001 developed by Kanovabiopharma, currently being evaluated in a Phase I trial. LNR 125.38 is another anti-IL-25 antibody developed by Lanier Biotherapeutics, currently at pre-clinical stage; LNR 125.38 significantly reduces type 2 cytokines and inflammatory cells increase in allergic mice and in mice with rhinovirus-induced asthma exacerbations. SM17 developed by SinoMab BioScience Limited is a first in-class humanized anti-IL17RB monoclonal antibody entering the Phase I clinical trial. SM17 does not block binding of IL-25 to IL-17RB but rather inhibits signal transduction via the IL-25/IL-17RB pathway. SM17 was humanized from its parent murine antibody D9.2, which demonstrated therapeutic potentials in pre-clinical murine studies for the treatment of inflammatory bowel disease, idiopathic pulmonary fibrosis, asthma and rhinovirus-induced asthma exacerbation.

Despite the demonstrated preclinical and clinical efficacies of these anti-alarmin antibodies (in particular, the anti-TSLP and anti-IL33 antibodies) in reducing both Th2 and non-Th2 inflammation, their therapeutic responses when used as add-on or monotherapy for the treatment of asthma and/or AD are somewhat suboptimal. A possible explanation is that TSLP, IL-33, and possibly IL-25 play redundant and overlapping functions/roles in the pathogenesis of asthma and AD. As such, blocking any single one of them is insufficient in achieving optimal clinical responses.

At the molecular level, these three alarmins all directly activate ILC2s, a member of the family of innate lymphoid cells (ILC). ILC2s are tissue-resident sentinels that respond rapidly to their environment through soluble inflammatory mediators, neurotrophic factors and cell-to-cell interactions. ILC2s have been shown to express receptors for IL-25, IL-33 and TSLP, and secrete IL-5 and IL-13 responding to these signals, which subsequently potentiate allergic responses. If ILC2s are dysregulated, they can contribute to over production of Th2 inflammatory cytokines leading to the development of allergic asthma, AD, allergic rhinitis, ulcerative colitis, and many chronic fibroproliferative disorders. ILC2s are also closely associated with rapid disease relapse in AD and OCS reliance in severe asthma.

In patients with severe eosinophilic asthma, a course of OCS resulted in a decrease in circulating eosinophils, Th2 cells, Tc2 cells, but not ILC2s. This suggests that ILC2s are more resistant to OCS compared to Th2 cells (Hynes, G., et al. 2018). Clinically, IL-13⁺ILC2s, when compared to Th2 cells, showed a stronger positive correlation with patient asthma control status and were more resistant to glucocorticoid-induced cell death and suppression of type 2 cytokine release [Jia, Yi et al. American Journal Of Respiratory Cell And Molecular Biology, vol. 55, 5 (2016): 675-683]. Recent studies further showed that three alarmins all played important roles in corticosteroid resistance in ILC2s. IL-33 was demonstrated in a murine model to rapidly increase the number of ILC2 in the peribronchial/perivascular region, while the pulmonary accumulation of ILC2 was dependent on CCL8-CCR8 signaling pathway. It is known that IL-33 treatment leads to the production of CCL8, predominantly from lung airway macrophages. Signaling via CCL8-CCR8 pathway in turn played critical roles for ILC2 cytokine (IL-13 and IL-5) production as well as IL-13′ activated ILC2 motility [Puttur, Franz et al. Science Immunology, vol. 4, 36 (2019): eaav7638]. A subpopulation of ILC2s known as “inflammatory” ILC2 (iILC2) was later found to be involved in the development of corticosteroid resistance. Correlation of disease severity and resistance to corticosteroid therapy was established in patients with chronic rhinosinusitis or asthma, especially when the number of circulating iILC2 and resident iILC2 in the inflamed mucosal tissue increased. Interestingly, the development and migration of iILC2s are IL-25 dependent [Miller, Mindy M et al. Science Immunology, vol. 5, 43 (2020): eaay3994; van der Ploeg, Esmee K et al. Science Immunology, vol. 6, 55 (2021): eabd3489]. TSLP treatment is also reported to increase steroid resistance of ILC2s. Bronchial alveolar lavage fluid (BALF) ILC2s harvested from asthmatic patients with high TSLP levels were steroid resistant. IL-7 and TSLP abrogated the inhibition of dexamethasone on type 2 cytokine production from blood ILC2s [Liu, Sucai et al. The Journal Of Allergy And Clinical Immunology, vol. 141, 1 (2018): 257-268.e6].

Alarmins also play critical roles in AD recurrence mediated by Th2 memory cells. The persisting skin-resident, treatment-resistant immune Th2 memory in AD has been identified in patients treated with dupilumab for one year. These memory Th2 cells are IL-13⁺, IL-17RB⁺, ST2⁺, TSLPR⁺, suggesting that they could respond to alarmins. Previous studies reported that IL-33 could induce Th2 memory cells to produce IL-31, a cytokine leading to severe pruritus in AD [Maier, Elisabeth et al. Journal of Immunology (Baltimore, Md.: 1950) vol. 193, 2 (2014): 645-54; Stott, Bryony et al. The Journal Of Allergy And Clinical Immunology, vol. 132, 2 (2013): 446-54.e5]. In another report, the infiltration of CRTH2⁺CD4⁺ Th2 memory cells into skin lesion of AD were associated with DCs activated by TSLP (TSLP-DCs) [Wang, Yui-I et al. Immunity, vol. 24, 6 (2006): 827-838]. Furthermore, IL-25 enhanced the proliferation of Th2 memory cell stimulated by TSLP-DCs [Wang, YIHsi et al. The Journal of Experimental Medicine, vol. 204, 8 (2007): 1837-47]. These studies indicated that neutralization of IL-4 and IL-13 by dupilumab might be insufficient to suppress the activity of Th2 memory cells. This may help to explain why considerable proportion of AD patients experience partial and non-durable responses when treated with Dupilumab [Bangert, Christine et al. Science Immunology, vol. 6, 55 (2021): eabe2749] [Wollenberg, A et al. The British Journal Of Dermatology, vol. 184, 3 (2021): 437-449]. Unlike IL-4 and IL-13, alarmins regulate both short-term Th2 effector functions and long-lasting Th2 memory in AD. Therefore, neutralization of alarmins serves as a novel approach for AD treatment.

As three alarmins play redundant and overlapping roles in type 2 immunity, blocking anyone of these 3 alarmins is unlikely to be sufficient in fully suppressing both Th2 and ILC2 activities. This may explain why Tezepelumab and Itepekimab only showed limited clinical efficacy on asthma and AD, despite that both Th2 and non-Th2 inflammatory responses are inhibited.

Albeit redundant, there are non-overlapping functions amongst these alarmins in Th2 as well as non-Th2 responses. IL-33 works in concert with IL-12 to directly induce the production of interferon gamma (IFNg) from human NK cells, a well-known disease-causing factor in the pathogenesis in inflammatory bowel disease (IBD). Studies in patient's biopsies have shown an increase in IL-33 levels in patients with active IBD, in particular ulcerative colitis (UC) [Kobori, Ayako et al. Journal of Gastroenterology, vol. 45, 10 (2010): 999-1007]. Additionally, UC-associated IL-33 is found in myofibroblasts, which tend to localize at the base of inflamed ulcerations in patients with UC [Sponheim, Jon et al. The American Journal Of Pathology, vol. 177, 6 (2010): 2804-15]. Blockade of IL-33/ST2 pathway was shown to ameliorate experimental colitis through enhancement of mucosal healing in mice and alleviate active disease in human, suggesting a pathogenic role of IL-33 in IBD [Sedhom, Mamdouh A K et al. Gut, vol. 62, 12 (2013): 1714-23]. In contrast, TSLP induces human DCs to express OX40 ligand (OX40L) but not IL-12, a preceding requisite for tregI naïve CD4+ T cells to produce IL-4, IL-5, and IL-13. Additionally, TSLP activated DCs produce chemokines such as CCL17/TARC and CCL22/MDC, which attract naïve T cells. TSLP stimulation of naïve CD4+ T cells, either directly via TSLPR or indirectly via engagement of OX40 Ligand (induced by TSLP on DC) with OX40 on T cells, induces a specialized Th2 polarization.

Interestingly, through DC activation, human TSLP and TLR3 ligands promote differentiation of Th17 cells with the central memory T cell phenotype. Similarly, IL-25 from skin localized mast cells stimulates dermal DCs to produce IL-1 and thereby contributes to activation of Th17 but not Th2 cells in the elicitation phase of contact dermatitis. In psoriatic skin, IL-25 stimulates the proliferation of keratinocytes and induces the production of inflammatory cytokines and chemokines, via activation of the STAT3 transcription factor. IL-25 expression in keratinocytes also contributes to the amplification of psoriasiform inflammation. Additionally, IL-25 was more potent than IL-33 in inducing IL-5 and IL-13 secretion from human peripheral blood mononuclear cells (PBMC) [Bartemes, Kathleen R et al. The Journal Of Allergy And Clinical Immunology, vol. 134, 3 (2014): 671-678.e4].

Although appearing redundant, three alarmins can play different roles in the regulation of ILC2, Th1, Th2, and Th17 activities and responses. In fact, they are reported to target different cell types in the central and peripheral systems, including but not limited to, basophils, macrophages, eosinophils, mast cells, fibroblasts and keratinocytes. Due to the heterogeneity of allergic diseases (e.g., contact dermatitis, AD, Th2-high/low asthma) and autoimmune diseases (e.g., inflammatory bowel disease, psoriasis), the blockade of a single alarmin may be effective only to a specific group of patients. For instance, Th1 and Th2 mixed phenotypes are most common in European-American AD, whereas Th17 and Th2 mixed phenotypes are the most common in Asian and pediatric AD [Renert-Yuval, Yael, and Emma Guttman-Yassky. Annals Of Allergy, Asthma & Immunology: Official Publication Of The American College Of Allergy, Asthma, & Immunology, vol. 124, 1 (2020): 28-35]. That leaves open rooms for improvement in addressing these immunological ailments and presents unmet medical needs for novel approaches and therapies catering to different subtypes of immunological diseases.

One approach to such alternative therapies may include the co-administration of biologics against two or more alarmins (e.g., antibodies) treating different aspects of the allergic disease (e.g., pediatric and adult AD). Co-administration requires either injections of two separate products or a single injection of a co-formulation of two different biologics. While two injections permit flexibility of dose amounts and timing, it is inconvenient to patients for compliance. Moreover, while a co-formulation might provide some flexibility of dose amounts, it is often quite challenging or impossible to find formulation conditions having acceptable viscosity (at relatively high concentration) and that promote chemical and physical stability due to different molecular characteristics of the two or more anti-alarmin biologics. Additionally, co administration and co-formulation involve the additive costs of two or more different drug therapies which can increase patient and/or payer costs. Thus, there remains a need for alternative therapies for treatment of allergic diseases that have disease modification and preferably such alternative therapies comprise a bispecific or multi-specific binding protein against different alarmins.

For illustrative purpose, the present invention provides a bispecific binding protein against 2 different alarmins; it can be in the form of bispecific protein with specificities against IL-17RB on one end, and against a soluble alarmin on the other end. Specifically, the bispecific protein can be an anti-IL17RB (receptor for IL-25) antibody, at the C-terminus of which either fused with (a) single chain Fv (scFv) targeting IL-33 or TSLP alarmins; or (b) the extracellular domain of the IL-33 receptor (ST2) or TSLP receptor (TSLPR). Although the present invention discloses the use of bispecific binding protein against an alarmin receptor and a different alarmin, the approach can be ramified into multi-specific binding proteins targeting different alarmin receptors, alarmins and other downstream cytokines.

Before the present disclosure is further described, it is to be understood that the disclosure is not limited to the particular embodiments set forth herein, and it is also to be understood that the terminology used herein is for the purpose of describing particular embodiments and is not intended to be limiting.

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, genetics and protein and nucleic acid chemistry and hybridization described herein are those well-known and commonly used in the art.

The term “a” or “an” entity refers to one entity; for example, “a vector,” is understood to represent one vector.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NO. It is understood that one skilled in the art can readily identify homologous sequences by reference to sequence sources, including but not limited to GenBank (ncbi.nlm.nih.gov/genbank/) and EMBL (embl.org/).

### 7.1 IL17RB Protein

Human IL17RB is a 47.9 kDa transmembrane protein (462 aa) that belongs to the IL-17 receptor family. IL-17RB is expressed in various endocrine tissues and in epithelial cells in different organs such as kidney and liver and mucosal tissues. Elevated IL-17RB expression is also found in lung tissues from asthmatic patients and in skin lesions from patients with AD. IL-17RB expression in human ILC2s, natural killer T (NKT) cells, and Th2 cells suggests a potential role in immune cells. IL-17RB is shared by 2 ligands: IL-17B and IL-25 (also known as IL-17E). IL-25 binds to the heterodimeric IL-17RA/IL-17RB complex while IL-17B is reported to bind to both heterodimeric receptor and IL-17RB homodimeric receptor [Wu, Heng-Hsiung et al. Science Translational Medicine, vol. 13,583 (2021): eabc2823]. The binding affinity (K_{D}) of IL-17B for IL-17RB is around 30-fold lower than that of IL-25 (IL-17E), with a similar association rate (Kₒₙ) but a substantially faster dissociation rate (K_{off}). Additional information about human IL-17RB, including its exemplary amino acid sequences can be found in public database such as GENEBANK (NCBI Ref. NP_061195.2). An exemplary sequence is also provided below.

(SEQ ID NO: 88)

| | |
|---|---|
| 1 | MSLVLLSLAA LCRSAVPREP TVQCGSETGP SPEWMLQHDL |
| | IPGDLRDLRV EPVTTSVATG |
| 61 | DYSILMNVSW VLRADASIRL LKATKICVTG KSNFQSYSCV |
| | RCNYTEAFQT QTRPSGGKWT |
| 121 | FSYIGFPVEL NTVYFIGAHN IPNANMNEDG PSMSVNFTSP |
| | GCLDHIMKYK KKCVKAGSLW |
| 181 | DPNITACKKN EETVEVNFTT TPLGNRYMAL IQHSTIIGFS |
| | QVFEPHQKKQ TRASVVIPVT |
| 241 | GDSEGATVQL TPYFPTCGSD CIRHKGTVVL CPQTGVPFPL |
| | DNNKSKPGGW LPLLLLSLLV |
| 301 | ATWVLVAGIY LMWRHERIKK TSFSTTTLLP PIKVLVVYPS |
| | EICFHHTICY FTEFLQNHCR |
| 361 | SEVILEKWOK KKIAEMGPVQ WLATQKKAAD KVVFLLSNDV |
| | NSVCDGTCGK SEGSPSENSQ |
| 421 | DLFPLAFNLF CSDLRSQIHL HKYVVVYFRE IDTKDDYNAL |
| | SVCPKYHLMK DATAFCAELL |
| 481 | HVKQQVSAGK RSQACHDGCC SL |

### 7.2 Bispecific Binding Proteins

The present disclosure provides bispecific binding proteins capable of specifically binding to two antigens. The binding proteins generally comprise variable light and variable heavy chain regions or domains that correspond to variable light and variable heavy chain regions or domains of immunoglobulins. At least one antigen binding moiety of the binding proteins is in a single chain format known in the art as a scFv. In some embodiments, the other antigen binding moiety comprises an IgG. In other embodiments, the other antigen binding moiety comprises a scFv.

In some embodiments, provided herein are methods and uses of antibody, or antigen-binding fragment thereof, that specifically bind to receptors for alarmins, alarmins, or both. The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, rabbit antibodies, camel antibodies, primate antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. In some embodiments, an antibody can comprise four polypeptide chains, two heavy (H) chains and two light (L) chains interconnected by disulfide bonds. Each heavy chain is comprised of a heavy chain variable region (abbreviated herein as HCVR or VH) and a heavy chain constant region. Each light chain is comprised of a light chain variable region (abbreviated herein as LCVR or VL) and a light chain constant region. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab (a monovalent fragment consisting of the VL, VH, CL and CH1 domains without the hinge region), Fab′ (a monovalent fragment consisting of the VL, VH, CL and CH1 domains attached with a hinge region), F(ab′)2 (a bivalent fragment comprising two Fab fragments linked by a disulfide bridge at the hinge region), Fd (a fragment consisting of the VH and CH1 domains), Fv (a fragment consisting of the VL and VH domains of a single arm of an antibody), linear antibodies, single chain antibody molecules (e.g., scFv, which is a single polypeptide chain having VL and VH regions joined by recombinant means), heavy chain antibodies (HCAbs), light chain antibodies (LCAbs), disulfide-linked scFv (dsscFv), diabodies (bivalent, bispecific antibodies), tribodies, tetrabodies, minibodies, dual variable domain antibodies (DVD), single variable domain antibodies (sdAbs or dAbs; e.g., camelid antibodies, alpaca antibodies), and single variable domain of heavy chain antibodies (VHH), and bispecific or multispecific antibodies formed from antibody fragments. A “bispecific” antibody or binding protein is an artificial hybrid antibody having two different antigen binding sites, which recognize and specifically bind two different targets. Bispecific binding antibodies and proteins can be produced by a variety of methods including fusion of hybridomas or linking of Fab′ fragments. See, e.g., Kostelny, S A et al. Journal of Immunology (Baltimore, Md.: 1950) vol. 148, 5 (1992): 1547-53; Songsivilai, S, and P J Lachmann. Clinical And Experimental Immunology, vol. 79, 3 (1990): 315-21.

The term “heavy chain” when used in reference to an antibody refers to a polypeptide chain of about 50-70 kDa, wherein the amino-terminal portion includes a variable region (VH) of about 120 to 130 or more amino acids and a carboxy-terminal portion that includes a constant region. In some embodiments, the heavy chain constant region is comprised of three domains, CH1, CH2 and CH3, and there is a short flexible hinge region connecting the CH1 and CH2 domains. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while and a contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The light chain constant region is comprised of one domain, CL. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. In some embodiments, each VH and VL is composed of three CDRs and four FRs, arranged from amino-terminus to carboxy-terminus in the following order: FR1, CDR1, FR2, CDR2, FR3, CDR3, FR4. Numbering of amino acid positions used herein is according to the EU Index, as in Kabat et al. (1991) Sequences of proteins of immunological interest. (U.S. Department of Health and Human Services, Washington, D.C.) 5^{th} ed.

A CDR refers to one of three hypervariable regions (H1, H2 or H3) within the non-framework region of the immunoglobulin (Ig or antibody) VH f-sheet framework, or one of three hypervariable regions (L1, L2 or L3) within the non-framework region of the antibody VL f-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat, E A et al. The Journal Of Biological Chemistry, vol. 252, 19 (1977): 6609-16.; Kabat, E A. Advances in protein chemistry vol. 32 (1978): 1-75.). The Chothia definition is based on the location of the structural loop regions, which defines CDR region sequences as those residues that are not part of the conserved f-sheet framework, and thus are able to adapt different conformations [Chothia, C, and A M Lesk. Journal of Molecular Biology, vol. 196, 4 (1987): 901-17]. Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures [Al-Lazikani, B et al. Journal Of Molecular Biology, vol. 273, 4 (1997): 927-48][Morea, V et al. Methods (San Diego, Calif.) vol. 20, 3 (2000): 267-79]. Because the number of residues within a hypervariable region varies in different antibodies, additional residues relative to the canonical positions are conventionally numbered with a, b, c and so forth next to the residue number in the canonical variable domain numbering scheme. Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDR1 | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDR1 | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest.

The terms “epitope” and “antigenic determinant” are used interchangeably herein and refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three-dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, FUNDAMENTAL IMMUNOLOGY SECOND EDITION, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less.

In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. K_{A} is the equilibrium association constant, which is also the reciprocal of the equilibrium dissociation constant, i.e., =1/K_{D}. For an antibody-antigen interaction, K_{D} can be calculated as the ratio of the products of concentrations of free antibody and free antigen over the concentrations of antibody-antigen complex, i.e., [antigen]×[antibody]/[antigen-antibody].

A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, Y et al. Journal of Molecular Biology, vol. 293, 4 (1999): 865-81). The K_{D} or K_{D} value can also be measured by using biolayer interferometry (BLI) using, for example, the Gator system (Probe Life), or the Octet-96 system (Sartorius, Gottingen, Germany). The K_{D} or K_{D} value can also be measured by using surface plasmon resonance assays by using a BIAcore system (e.g., Pharmacia Biosensor AB, Uppsala, Sweden and Piscataway, NJ).

The term “variant” as used herein in relation to a protein or a polypeptide with particular sequence features (the “reference protein” or “reference polypeptide”) refers to a different protein or polypeptide having one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The terms “polynucleotide,” “nucleic acid,” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid molecule can be single-stranded or double-stranded.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure. In some embodiments, a polypeptide, peptide, protein, antibody, polynucleotide, vector, cell, or composition which is isolated is substantially free of other cellular material and/or chemicals.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” as used herein, is one in which one amino acid residue is replaced with another amino acid residue having a similar side chain. Amino acid or residue that is “conservatively similar” as used herein refers to non-identical amino acid residue having similar side chains. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an IgG consisting bispecific binding proteins), and it is further understood that expression levels can be optimized to obtain sufficient expression using methods well known in the art.

As used herein, the term “host cell” refers to a cell into which a genetical material, such as a recombinant expression vector can be introduced or has been introduced. Host cells include not only the subject cell introduced with the exogenous genetic material, but also the progeny of such a cell. Because certain modifications may occur in succeeding generations due to either mutation or environmental influences, such progeny may not be identical to the parent cell.

As used herein and understood in the art, “EC” means effective concentration of an agent (e.g., antibody), and is commonly used in dose-response curves. The “effect” of the agent can be a positive (activatory) effect or a negative effect. The term “EC50” refers to the concentration of an active agent (e.g., antibody) that gives half-maximal response. Also as used herein and understood in the art, “IC” means concentration of an agent that has an inhibitory effect, and is also commonly used for dose-response curves. The term “IC50” refers to the concentration of an agent (e.g., antibody) where the activity that it inhibits is reduced by half.

As described in the section above, a bispecific binding protein can comprise two polypeptides linked together via disulfide bonds, with each polypeptide comprising a first scFv region and a second scFv region at its respective N- and C-termini. The two scFv regions specifically bind different antigens. Between the first and second scFv regions of each polypeptide are sequences comprising the hinge, CH2, and CH3 domains of immunoglobulins.

Alternatively, a bispecific binding protein can comprise an IgG that has a scFv region linked at the C-terminal end of each CH3 domain. In this case, the variable heavy and light chains in the Fab regions of the IgG specifically bind one antigen, and the C-terminal scFv regions comprise different variable heavy and light chains that specifically bind a second antigen.

As noted above, a bispecific binding protein can bind to two different antigens via two domains, one at the N-terminal end that binds antigen X and the other at the C-terminal end that binds antigen Y. In some embodiments, the bispecific binding protein binds the first domain of an alarmin receptor and the second domain of an alarmin. In one particular embodiment, the bispecific binding protein suppresses IL17RB activation by binding via the first domain and binds TSLP via the second domain. In another embodiment, the bispecific binding protein suppresses IL17RB activation by binding via the first domain and binds to IL33 via the second domain.

In one embodiment, a bispecific binding protein comprises two polypeptides of formula:

X-H-Fc-L-scFv^{Y}

wherein X is scFv^{X} or an Fab region, wherein X specifically binds a first antigen and scFv specifically binds a second antigen, H is a hinge region, Fc comprises CH2 and CH3 regions of an immunoglobulin, scFv^{X} and scFv^{Y} are each independently a single chain variable fragment, and L is a polypeptide linker. In some embodiments, one of the two antigens is an immunomodulatory protein and the other is an alarmin. In some embodiments, when X is an Fab region, then the first antigen is an immunomodulatory protein and the other is an alarmin. In some embodiments, when X is an Fab region, then the first antigen an alarmin and the other is an immunomodulatory protein.

In an embodiment, a bispecific binding protein comprises two polypeptides of formula:

X-H-Fc-L-ECD^{Y}

wherein X is scFv^{X} or an Fab region, wherein X specifically binds a first antigen and ECD^{Y} is the extracellular domain of alarmin receptor, H is a hinge region, Fc comprises CH2 and CH3 regions of an immunoglobulin, scFv^{X} is a single chain variable fragment and L is a polypeptide linker. In some embodiments, one of the two antigens is an immunomodulatory protein and the other is the extracellular domain of alarmin receptor. In some embodiments, when X is an Fab region, then the first antigen is an immunomodulatory protein and the other is an alarmin. In some embodiments, ECD^{Y} is TSLP receptor extracellular domain. In some embodiments, ECD^{Y} is IL-33 receptor extracellular domain.

The VH and VL chains incorporated into the bispecific binding proteins may be derived from multiple sources, including pre-existing antibodies, newly generated antibodies, and VH and VL chain libraries. Specific exemplary embodiments of VH and VL chains that may be incorporated into bispecific binding proteins, as well as specific exemplary embodiments of bispecific binding proteins that compete for binding an immunomodulatory protein, such as IL17RB or TSLPR, are provided in the Detailed Description section.

Nucleic acids comprising nucleotide sequences encoding the polypeptides of the disclosure are provided herein. Methods of producing polypeptides, culturing host cells and recovering the polypeptides are also provided and discussed further in the Detailed Description below.

In another aspect, the present disclosure provides compositions including the bispecific binding proteins described herein. The compositions generally comprise one or more bispecific binding proteins as described herein, and/or salts thereof, and one or more excipients, carriers or diluents.

Current anti-alarmin antibody therapies have limited effects on AD and moderate-severe asthma. Specifically, Phase II AD clinical trials of Tezepelumab, an anti-TSLP antibody, and Etokimab, an anti-IL-33 antibody, failed to meet their respective primary end-points. However, for the treatment of asthma, both Tezepelumab and Itepekimab met their primary end points in both Phase II and Phase III trials, regardless of their limited therapeutic effects: Tezepelumab failed to reduce the daily dose of OCS usage, and Itepekimab was comparatively inferior to Dupilumab. The bispecific proteins described herein bind specifically to two antigens, such as an alarmin receptor and an alarmin, and block immune responses induced by two different alarmins. Such inhibition of alarmin-induced responses potentially allows for less frequent dosing and a longer-lasting disease remission in allergic disease or disorder.

In some embodiments, provided herein are uses of a bispecific binding protein thereof that specifically binds to alarmins and/or receptors for alarmins for the preparation of a medicament for the reduction of OCS daily dose in the treatment of Th2-high and/or Th2-low immunological disorders such as asthma. In the uses described above, the bispecific binding protein (a) blocks both IL-25 signaling and TSLP signaling and/or (b) blocks both IL-25 signaling and IL-33 signaling. In some embodiments, the bispecific binding protein thereof specifically binds to human IL-17RB and TSLP. In some embodiments, the bispecific binding protein thereof specifically binds to human IL-17RB and IL-33.

Provided herein are also methods of treating an allergic disease or disorder in a subject in need thereof that comprise administering to the subject a therapeutically effective amount of a bispecific binding protein thereof that specifically binds to IL-17RB and TSLP, or IL-17RB and IL-33, wherein the bispecific binding protein (a) blocks both IL-25 signaling and TSLP signaling and/or (b) blocks both IL-25 signaling and IL-33 signaling. In some embodiments, bispecific binding protein thereof specifically binds to human IL-17RB and TSLP, or human IL-17RB and IL-33. In some embodiments, the allergic disease or disorder is AD and asthma. In some embodiments, allergic disease or disorder is pre-clinical AD and asthma.

As used herein, the term “treat” and its grammatical equivalents in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to AD, the term “treat” and its grammatical equivalents refer to an action that reduces the severity of the disease, or retards or slows the progression of the disease, including, but not limited to (a) reducing the frequency of dosing to achieve disease remission, or decrease the incidence of disease relapse, or (b) delaying, ameliorating or minimizing one or more symptoms associated with AD, quantified by the Eczema Area and Severity Index (EASI), (c) reducing the amount and frequency of OCS use.

The term “block” and its grammatical equivalents refer to an action that reduces the biological function of alarmin in a way including but not limited to (a) directly competing for the binding site of alarmin on its corresponding receptors, or (b) preventing heterodimerization of corresponding receptors, reducing biological effects induced by alarmin occupancy.

As used herein, the term “administer” and its grammatical equivalents refer to the act of delivering, or causing to be delivered, a therapeutic or a pharmaceutical composition to the body of a subject by a method described herein or otherwise known in the art. The therapeutic can be a compound, a polypeptide, or a cell. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); subcutaneous (SC), transdermal dosage forms, including creams, jellies, powders, or patches; buccal dosage forms; inhalation powders, sprays, suspensions, and rectal suppositories.

As used herein, the terms “effective amount,” “therapeutically effective amount,” and their grammatical equivalents refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. A therapeutically effective amount is also one in which any toxic or detrimental effects of the therapeutic agent are outweighed by the therapeutically beneficial effects. An appropriate “effective amount” in any individual case can vary according to factors such as the disease state, age, sex, and weight of the individual, and can be determined by one of ordinary skill in the art using routine experimentation. A “prophylactically effective amount” refers to an amount effective, at dosages and for periods of time necessary, to achieve the desired prophylactic result, for example, the delay or prevention of the onset of a disease or disorder. Typically, since a prophylactic dose is used in subjects prior to or at an earlier stage of disease, the prophylactically effective amount is commonly less than the therapeutically effective amount.

The term “subject” as used herein refers to any animal (e.g., a mammal), including, but not limited to, humans, non-human primates, canines, felines, rodents, and the like, which is to be the recipient of a particular treatment.

In some embodiments, the bispecific binding proteins disclosed herein (a) inhibit IL5/CCL17/IL13/CCL8/IFNγ secretion from human PBMC/or (b) inhibit ILC2 migration and proliferation. As such, the bispecific binding proteins disclosed herein demonstrate long-lasting efficacy by directly suppressing ILC2 activity. Moreover, methods disclosed herein have the additional therapeutic benefit of reducing Th2 and ILC2 cell migration. In some embodiments, provided herein are methods of treating OCS resistant-associated disease or disorder using anti-IL17RB/anti-TSLP or anti-IL17RB/anti-IL33 bispecific binding proteins disclosed herein.

Methods provided herein can treat an allergic disease or disorder. The allergic disease or disorder can be clinical or pre-clinical allergic asthma, allergic rhinosinusitis, allergic conjunctivitis, or AD.

Although the above indications are preferred, other diseases, disorders, or conditions may be amenable to treatment with or may be prevented by administration of a bispecific antigen binding protein as disclosed herein to a subject. Such diseases, disorders, and conditions include, but are not limited to, inflammation, autoimmune disease, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodosa, Wegener's granulomatosis, arteritis, polymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, AD, atherosclerosis, lupus, Still's disease, Systemic Lupus Erythematosus (SLE), myasthenia gravis, inflammatory bowel disease 'IBD), Crohn's disease, ulcerative colitis, celiac disease, multiple sclerosis (MS), asthma, COPD, Guillain-Barre disease, Type I diabetes mellitus, Graves' disease, Addison's disease, Raynaud's phenomenon, autoimmune hepatitis, GVHD, and the like. In specific embodiments, pharmaceutical compositions comprising a therapeutically effective amount of anti-IL17RB/anti-TSLP bispecific binding proteins are provided. In another specific embodiments, pharmaceutical compositions comprising a therapeutically effective amount of anti-IL17RB/anti-IL33 bispecific binding proteins are provided.

Based on the data presented herein, treating subjects with allergic disease or disorder with bispecific binding proteins described herein is expected to provide therapeutic benefits.

### 7.2.1 Exemplary Anti-IL17RB/Anti-Alarmin Bispecific Binding Proteins

The present invention provides bispecific binding proteins comprising an immunoglobulin G antibody (IgG) with specificity against an alarmin receptor that is fused at each of the C-terminus of an immunoglobulin chain to either (a) a single chain variable fragment (scFv) specific to a particular alarmin, or (b) an extracellular domain of an alarmin receptor.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 7, the amino acid sequence of HCDR5 is SEQ ID NO: 8, the amino acid sequence of HCDR6 is SEQ ID NO: 9, the amino acid sequence of LCDR4 is SEQ ID NO: 10, the amino acid sequence of LCDR5 is SEQ ID NO: 11, and the amino acid sequence of LCDR6 is SEQ ID NO: 12, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 36, the amino acid sequence of HCDR5 is SEQ ID NO: 37, the amino acid sequence of HCDR6 is SEQ ID NO: 38, the amino acid sequence of LCDR4 is SEQ ID NO: 39, the amino acid sequence of LCDR5 is SEQ ID NO: 40, and the amino acid sequence of LCDR6 is SEQ ID NO: 41, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 42, the amino acid sequence of HCDR5 is SEQ ID NO: 43, the amino acid sequence of HCDR6 is SEQ ID NO: 44, the amino acid sequence of LCDR4 is SEQ ID NO: 45, the amino acid sequence of LCDR5 is SEQ ID NO: 46, and the amino acid sequence of LCDR6 is SEQ ID NO: 47, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 13, the amino acid sequence of HCDR5 is SEQ ID NO: 14, the amino acid sequence of HCDR6 is SEQ ID NO: 15, the amino acid sequence of LCDR4 is SEQ ID NO: 16, the amino acid sequence of LCDR5 is SEQ ID NO: 17, and the amino acid sequence of LCDR6 is SEQ ID NO: 18, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 48, the amino acid sequence of HCDR5 is SEQ ID NO: 49, the amino acid sequence of HCDR6 is SEQ ID NO: 50, the amino acid sequence of LCDR4 is SEQ ID NO: 51, the amino acid sequence of LCDR5 is SEQ ID NO: 52, and the amino acid sequence of LCDR6 is SEQ ID NO: 53, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and two scFv wherein, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 54, the amino acid sequence of HCDR5 is SEQ ID NO: 55, the amino acid sequence of HCDR6 is SEQ ID NO: 56, the amino acid sequence of LCDR4 is SEQ ID NO: 57, the amino acid sequence of LCDR5 is SEQ ID NO: 58, and the amino acid sequence of LCDR6 is SEQ ID NO: 59, wherein each scFv is linked at the N-terminus of HCVR2 of each scFv to said IgG antibody at the C-terminus of each IgG HC via a polypeptide linker (L1), and wherein the HCVR2 of each scFv is linked at the C-terminus of the HCVR2 to the LCVR2 of the same scFv at the N-terminus of the LCVR2 of the same scFv via a second polypeptide linker (L2). In some embodiments, a HCDR variant thereof has up to about 3, about 5, about 8, about 10, about 12, or about 15 amino acid substitutions, additions, and/or deletions in the SEQ ID NO:1-3. In some embodiments, a LCDR variant thereof has up to about 3, about 5, about 8, about 10, about 12, or about 15 amino acid substitutions, additions, and/or deletions in the SEQ ID NO:4-6.

In some embodiments, the present invention provides a bispecific antibody comprising an IgG and extracellular domain of alarmin receptor, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) said extracellular domain of alarmin receptor, wherein the amino acid sequence is SEQ ID NO: 19 or SEQ ID NO: 34, wherein each alarmin extracellular domain is linked at the C-terminus of amino acid sequence of each domain to said IgG antibody at the C-terminus of each IgG HC via a polypeptide linker (L1). In some embodiments, a HCDR variant thereof has up to about 3, about 5, about 8, about 10, about 12, or about 15 amino acid substitutions, additions, and/or deletions in the SEQ ID NO:1-3.

In some embodiments, the present invention provides a bispecific antibody comprising an IgG and extracellular domain of alarmin receptor, (a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6; and (b) said extracellular domain of alarmin receptor, wherein the amino acid sequence is SEQ ID NO: 20 or SEQ ID NO: 35, wherein each alarmin extracellular domain is linked at the C-terminus of amino acid sequence of each domain to said IgG antibody at the C-terminus of each IgG HC via a polypeptide linker (L1). In some embodiments, a HCDR variant thereof has up to about 3, about 5, about 8, about 10, about 12, or about 15 amino acid substitutions, additions, and/or deletions in the SEQ ID NO:1-3.

In some embodiments, a bispecific binding protein of the disclosure comprises polypeptide linkers (L1 and/or L2), the linker having a sequence corresponding to a sequence selected from one of the sequences in the table below:

| Sequence (N→C) | Identifier |
|---|---|
| VDGASSPVNVSSPSVQDI | SEQ ID NO: 21 |
| VDGASSPVNVGSPSVQDI | SEQ ID NO: 22 |
| GASSPVNVSSPSV | SEQ ID NO: 23 |
| GGGGSGGGNGTGSGGGGS | SEQ ID NO: 24 |
| LSAGGHGGLDNDTSAFHL | SEQ ID NO: 25 |
| SDKTHTSPPSPAPESSGG | SEQ ID NO: 26 |
| VTTTDFQIQTEMAATMET | SEQ ID NO: 27 |
| DFLPTTAQPTKKSTLKKR | SEQ ID NO: 28 |
| TESRSPPAENEVSTPMQA | SEQ ID NO: 29 |
| GGGGSGGGNGSGSGGGGS | SEQ ID NO: 30 |
| GGGGSGGGSGGGSGGGGS | SEQ ID NO: 31 |

In a further embodiment of the bispecific binding protein of the present invention, the amino acid sequence of each HC is SEQ ID NO: 32, the amino acid sequence of each LC is SEQ ID NO: 33. In a still further embodiment of the bispecific binding proteins of the present invention, polypeptide linker L1 has a sequence of SEQ ID NO: 30.

In some embodiment of the bispecific binding protein of the present invention, the amino acid sequence of each LC is SEQ ID NO: 33. In a further embodiment, the amino acid sequence of each HC could be anyone selected from SEQ ID NO: 60-74.

In some embodiments, the anti-IL17RB IgG consisting bispecific binding proteins that can be used in methods disclosed herein is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In certain embodiments, the antibody comprises a heavy chain constant region, such as an IgG1, IgG2, IgG3, IgG4, IgA, IgE, IgM or IgD constant region or any of the above constant region with the glycosylation site and/or the glycoforms at the glycosylation site modified.

In some embodiments, the anti-IL17RB binding portion of the bispecific binding proteins used in methods disclosed herein is in the form of a single domain antibody (sdAb), a heavy chain antibody (HCAb), a Fv, a single-chain variable fragment (scFv), or a (scFv)2 fused to the constant regions of an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In certain embodiments, the antibody comprises a heavy chain constant region, such as an IgG1, IgG2, IgG3, IgG4, IgA, IgE, IgM or IgD constant region or any of the above constant region with the glycosylation site and/or the glycoforms at the glycosylation site modified.

In some embodiments, used in methods disclosed herein are antigen-binding fragments of an anti-alarmin antibody. In some embodiments, antigen-binding fragments provided herein can be a single domain antibody (sdAb), a heavy chain antibody (HCAb), an Fab, an Fab′, an F(ab′)2, an Fv, a single-chain variable fragment (scFv), or an (scFv)2. In some embodiments, the antigen-binding fragment of an anti-alarmin antibody is a single domain antibody (sdAb). In some embodiments, the antigen-binding fragment of an anti-alarmin antibody is a heavy chain antibody (HCAb). In some embodiments, the antigen-binding fragment of an anti-alarmin antibody is an Fab. In some embodiments, the antigen-binding fragment of an anti-alarmin antibody is an Fab′. In some embodiments, the antigen-binding fragment of an anti-alarmin antibody is a F(ab′)2. In some embodiments, the antigen-binding fragment of an anti-alarmin antibody is a Fv. In some embodiments, the antigen-binding fragment of an anti-alarmin antibody is a scFv. In some embodiments, the antigen-binding fragment of an anti-alarmin antibody is a disulfide-linked scFv [(scFv)2]. In some embodiments, the antigen-binding fragment of an anti-alarmin antibody is a diabody (dAb). In a further embodiment, the anti-alarmin antigen-binding fragment neutralizes the activities of the alarmins, including IL-25, IL-33 and TSLP.

The term “activity” includes properties such as the ability to bind a target protein with specificity, the affinity of an antibody or binding protein for a protein, the ability to neutralize the biological activity of a target protein, the ability to inhibit interaction of a target protein with its natural receptor (s) or natural ligand (s), and the like.

In some embodiments, used in methods disclosed herein are recombinant anti-IL17RB/anti-TSLP bispecific antibodies. In some embodiments, used in methods disclosed herein are recombinant anti-IL17RB/anti-IL33 bispecific antibodies. In some embodiments, used in methods disclosed herein are recombinant anti-IL17RB/TSLP receptor bispecific binding proteins. In some embodiments, used in methods disclosed herein are recombinant anti-IL17RB/IL33 receptor bispecific binding proteins.

In some embodiments, the IgG consisting bispecific binding proteins provided herein is chimeric antibody. In some embodiments, the IgG consisting bispecific binding proteins provided herein is humanized antibody. In some embodiments, the IgG consisting bispecific binding proteins provided herein is human antibody. In some embodiments, the scFv consisting bispecific binding proteins provided herein is chimeric scFv. In some embodiments, the scFv consisting bispecific binding proteins provided herein is humanized scFv. In some embodiments, the scFv consisting bispecific binding proteins provided herein is human scFv. In some embodiments, bispecific binding proteins used in the methods provided herein are isolated. In some embodiments, bispecific binding proteins used in the methods provided herein are substantially pure.

Various methods for generating humanized antibodies and scFv are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant IL17RB or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant IL17RB or an epitope thereof).

Human antibodies and scFv can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, the IgG consisting bispecific binding proteins used in methods provided herein comprises a monovalent antigen-binding site. In some embodiments, the IgG consisting bispecific binding proteins comprises a monospecific binding site. In some embodiments, the IgG consisting bispecific binding proteins comprises a bivalent binding site. In some embodiments, the scFv consisting bispecific binding proteins used in methods provided herein comprises a monovalent antigen-binding site. In some embodiments, the scFv consisting bispecific binding proteins comprises a monospecific binding site. In some embodiments, the scFv consisting bispecific binding proteins comprises a bivalent binding site.

In some embodiments, the IgG consisting bispecific binding proteins used in methods provided herein is SM17. The term “SM17” refers to a humanized antibody against human IL17RB (hIL17RB). Sequence features of SM17 are provided in the table below. Additional description of the structural and functional features of SM17 can be found in WO2020115319A1, incorporated herein in their entirety by reference. The HC sequence of SM17 is SEQ ID NO: 32. The LC sequence of SM17 is SEQ ID NO: 33. The amino acid sequence of SM17 HCDR1 is SEQ ID NO: 1, the amino acid sequence of SM17 HCDR2 is SEQ ID NO: 2, the amino acid sequence of SM17 HCDR3 is SEQ ID NO: 3, the amino acid sequence of SM17 LCDR1 is SEQ ID NO: 4, the amino acid sequence of SM17 LCDR2 is SEQ ID NO: 5, and the amino acid sequence of SM17 LCDR3 is SEQ ID NO: 6.

In some embodiments, the IgG consisting bispecific binding proteins that can be used in methods provided herein comprise one, two, three, four, five, and/or six CDRs of SM17. In some embodiments, the IgG consisting bispecific binding proteins comprise a VL comprising one, two, and/or three, VL CDRs of SM17. In some embodiments, the IgG consisting bispecific binding proteins provided herein comprise a VH comprising one, two, and/or three VH CDRs of SM17. In some embodiments, the IgG consisting bispecific binding proteins provided herein comprise one, two, and/or three VL CDRs and one, two, and/or three VH CDRs of SM17.

It is well known in the art that VH CDR3 and VL CDR3 domains play an important role in the binding specificity/affinity of an antibody for an antigen. Accordingly, in some embodiments, the IgG consisting bispecific binding proteins thereof that can be used in methods disclosed herein can have the appropriate association/dissociation kinetics with human IL17RB and have the VH CDR3 and VL CDR3 that are structurally identical to or related to those of SM17. A consensus motif for the SM17 VL CDR3 comprising the amino acid sequence SEQ ID NO:6 can be modified by substituting one or more of the amino acid(s) to adjust the antibody affinity without changing its binding specificity, or alternatively be replaced by the VL CDR3 of an irrelevant human antibody that exhibits sufficient similarities to the SM17 VL CDR3 using criteria as described in Chinese Pat. No. ZL200880024788.2, which is incorporated herewith by reference. Similarly, a consensus motif for the SM17 VH CDR3 comprising the amino acid sequence SEQ ID NO:3 can be modified by substituting one or more of the amino acid(s) to adjust the antibody affinity without changing its binding specificity, or alternatively be replaced by the VH CDR3 of an irrelevant human antibody that exhibits sufficient similarities to the SM17 VH CDR3 using criteria as described in Chinese Pat. No. ZL200880024788.2, which is incorporated herewith by reference.

The skilled artisan would appreciate that certain substitution(s) of amino acids within the CDR3 domains would not change the epitope specificity of the antibody, in particular substitutions with conservative amino acids. As such, in some embodiments, the CDR3 of the antibodies or antigen-binding fragments provided herein (e.g., SM17 or) can be replaced with the CDR3 from a human or primate antibody that (1) is identical in the number of residues and exhibits 50% or higher sequence homology to the SM17 CDR3, (2) contains at least one, preferably more, aromatic residue(s) that is (are) identical or conservatively similar to the residue(s) at corresponding position(s) in the SM17 CDR3, (3) contains at least one, preferably more, charged residue(s) that is (are) identical or conservatively similar to the residue(s) at corresponding position(s) in the SM17 CDR3, and/or (4) contains at least one, preferably more, amino acid residue(s) that is/are identical or conservatively similar to the residue(s) at corresponding position(s) in the SM17 CDR3 at positions that are known to be important for maintaining the binding site structure/contacts of the anti-IL17RB antibody as determined by crystal structure and/or computer database analysis. In some embodiments, no more than one to five conservative amino acid substitutions are made with the SM17 VL and/or VH CDR3 domains, or VL and/or VH CDR3 from irrelevant primate or human antibodies containing no more than one to five conservatively similar residues are used to replace the VL and/or VH CDR3 of SM17. In some embodiments, no more than one to three conservative amino acid substitutions are made within the SM17 VL and/or VH CDR3 domains, or VL and/or VH CDR3 from irrelevant primate or human antibodies containing no more than one to three conservatively similar residues is used to replace the VL and/or VH CDR3 of SM17.

In some embodiments, the IgG consisting bispecific binding proteins used in methods provided herein specifically binds to IL17RB comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:33. In some embodiments, the IgG consisting bispecific binding proteins used in methods provided herein specifically binds to IL17RB comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:32. In some embodiments, the IgG consisting bispecific binding proteins used in methods provided herein specifically binds to IL17RB comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to a the amino acid sequence of SEQ ID NO:33; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to the amino acid sequence of SEQ ID NO:32.

In some embodiments, the IgG consisting bispecific binding proteins used in methods provided herein specifically binds to IL17RB comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:33. In some embodiments, the IgG consisting bispecific binding proteins used in methods provided herein has a VL having at least 85% sequence identity to SEQ ID NO:33. In some embodiments, the IgG consisting bispecific binding proteins used in methods provided herein has a VL having at least 90% sequence identity to SEQ ID NO:33. In some embodiments, the IgG consisting bispecific binding proteins used in methods provided herein has a VL having at least 95% sequence identity to SEQ ID NO:33. In some embodiments, the IgG consisting bispecific binding proteins used in methods provided herein has a VL having at least 98% sequence identity to SEQ ID NO:33. In some embodiments, the IgG consisting bispecific binding proteins used in methods provided herein that specifically bind to IL17RB comprising a VL having the amino acid sequence of SEQ ID NO:33.

In some embodiments, the IgG consisting bispecific binding proteins used in methods provided herein specifically binds to IL17RB comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:32. In some embodiments, the IgG consisting bispecific binding proteins used in methods provided herein has a VH having at least 85% sequence identity to SEQ ID NO:32. In some embodiments, the IgG consisting bispecific binding proteins used in methods provided herein has a VH having at least 90% sequence identity to SEQ ID NO:32. In some embodiments, the IgG consisting bispecific binding proteins used in methods provided herein has a VH having at least 95% sequence identity to SEQ ID NO:32. In some embodiments, the IgG consisting bispecific binding proteins used in methods provided herein has a VH having at least 98% sequence identity to SEQ ID NO:32. In some embodiments, the IgG consisting bispecific binding proteins used in methods provided herein that specifically bind to IL17RB comprising a VH having the amino acid sequence of SEQ ID NO:32.

In some embodiments, the IgG consisting bispecific binding proteins thereof that specifically binds to IL17RB comprising a VL and a VH, wherein the VL and VH have the amino acid sequences of SEQ ID NOs: 33 and 32, respectively. the IgG consisting bispecific binding proteins thereof that specifically binds to IL17RB can comprise a combination of any VL disclosed herein and any VH disclosed herein.

In some embodiments, the IgG consisting bispecific binding proteins thereof that specifically binds to IL17RB comprising (a) a VL comprising VL CDRs 1, 2, and 3 from a VL having the amino acid sequence of SEQ ID NO:33; and/or (b) a VH comprising VH CDRs 1, 2, and 3 from a VH having the amino acid sequence of SEQ ID NO: 32.

In some embodiments, the IgG consisting bispecific binding proteins thereof that specifically binds to IL17RB comprising a VL, wherein the VL comprises VL CDRs 1, 2, and 3 from a VL having the amino acid sequence of SEQ ID NO: 33.

In some embodiments, the IgG consisting bispecific binding proteins thereof that specifically binds to IL17RB comprising a VH, wherein the VH comprises VH CDRs 1, 2, and 3 from a VH having the amino acid sequence of SEQ ID NO:32.

In some embodiments, the IgG consisting bispecific binding proteins thereof that specifically binds to IL17RB comprising a VL and a VH, wherein the VL comprises VL CDR1, CDR2, and CDR3 from a VL having the amino acid sequence of SEQ ID NO:33, and the VH comprises VH CDR1, CDR2, and CDR3 from a VH having the amino acid sequence of SEQ ID NO:32.

In some embodiments, the IgG consisting bispecific binding proteins thereof provided herein is a variant of SM17. The SM17 variant can have a VL that is a variant of the VL of SM17 having up to about 5 amino acid substitutions, additions, and/or deletions in SEQ ID NO:33. The SM17 variant can have a VH that is a variant of the VH of SM17 having up to about 5 amino acid substitutions, additions, and/or deletions in SEQ ID NO:32. The amino acid substitutions, additions, and/or deletions can be in the VH CDRs or VL CDRs. In some embodiments, the amino acid substitutions, additions, and/or deletions are not in the CDRs. In some embodiments, the variant of SM17 has up to about 5 conservative amino acid substitutions. In some embodiments, the variant of SM17 has up to 3 conservative amino acid substitutions.

In some embodiments, the IgG consisting bispecific binding proteins that can be used in methods disclosed herein comprise a VH or VL that has at least one framework (FR) region. In some embodiments, the FR one (FR1) regions for VL can be from the VκID human germline family, the FR two (FR2) regions for VL can be from the Vκ1 human germline family, the FR three (FR3) regions for VL can be from the Vκ1 human germline family, and the FR four (FR4) regions for VL can be from the VκJ1 human germline family. In some embodiments, the FR1, FR2, FR3, and FR4 for VL can have the framework sequences shown in WO2020115319A1 that are incorporated by reference herein. In some embodiments, the framework one (FR1) regions for VH can be from the V_{H}3 human germline family; the framework two (FR2) regions for VH can be from the V_{H}3 human germline family; the framework three (FR3) regions for VH can be from the V_{H}3 human germline family; and the framework four (FR4) regions for VH can be from the V_{H}J5 human germline family. In some embodiments, FR1, FR2, FR3, and FR4 for VH can have the framework sequences shown in WO2020115319A1 referenced above and incorporated by reference herein.

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

It is known in the art that the constant region(s) of an antibody mediates several effector functions, and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are several Fc receptors which are specific for different classes of antibody, including IgG (gamma receptors), IgE (epsilon receptors), IgA (alpha receptors) and IgM (mu receptors). Binding of antibody to Fc receptors on cell surfaces triggers many important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production. In some embodiments, the bispecific binding proteins described herein comprise at least one constant region of a human IgA antibody. In some embodiments, the bispecific binding proteins described herein comprise at least one constant region of a human IgD antibody. In some embodiments, bispecific binding proteins described herein comprise at least one constant region of a human IgE antibody. In some Embodiments, the bispecific binding proteins described herein comprise at least one constant region of a human IgG antibody. In some embodiments, the bispecific binding proteins described herein comprise at least one constant region of a human IgM antibody. In some embodiments, the bispecific binding proteins described herein comprise at least one constant region of a human IgG1 antibody. In some embodiments, the bispecific binding proteins described herein comprise at least one constant region of a human IgG2 antibody. In some embodiments, the bispecific binding proteins described herein comprise at least one constant region of a human IgG3 antibody. In some embodiments, the bispecific binding proteins described herein comprise at least one constant region of a human IgG4 antibody.

In some embodiments, at least one or more of the constant regions has been modified or deleted in the bispecific binding proteins described herein. In some embodiments, the bispecific binding proteins comprise modifications to one or more of the three heavy chain constant regions (CH1, CH2 or CH3) and/or to the light chain constant region (CL). In some embodiments, the heavy chain constant region of the modified bispecific binding proteins comprises at least one human constant region. In some embodiments, the heavy chain constant region of the modified bispecific binding proteins comprises more than one human constant region. In some embodiments, modifications to the constant region comprise additions, deletions, or substitutions of one or more amino acids in one or more regions.

In some embodiments, one or more regions are partially or entirely deleted from the constant regions of the modified bispecific binding proteins. In some embodiments, the entire CH2 domain has been removed from a bispecific binding protein (ΔCH2 constructs). In some embodiments, a deleted constant region is replaced by a short amino acid spacer that provides some of the molecular flexibility typically imparted by the absent constant region. In some embodiments, a modified bispecific binding protein comprises a CH3 domain directly fused to the hinge region of the bispecific binding protein. In some embodiments, a modified bispecific binding protein comprises a peptide spacer inserted between the hinge region and modified CH2 and/or CH3 domains.

In some embodiments, the bispecific binding proteins comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified bispecific binding proteins (e.g., modified Fc region) provide for altered effector functions that, in turn, affect the biological profile of the bispecific binding protein. For example, in some embodiments, the deletion or inactivation (through point mutations or other means) of a constant region reduces Fc receptor binding of the modified bispecific binding protein as it circulates. In some embodiments, the constant region modifications reduce the immunogenicity of the bispecific binding protein. In some embodiments, the constant region modifications increase the serum half-life of the bispecific binding protein. In some embodiments, the constant region modifications reduce the serum half-life of the bispecific binding protein. In some embodiments, the constant region modifications decrease or remove ADCC and/or complement dependent cytotoxicity (CDC) of the bispecific binding protein. In some embodiments, specific amino acid substitutions in a human IgG1 Fc region with corresponding IgG2 or IgG4 residues reduce effector functions (e.g., ADCC and CDC) in the modified bispecific binding protein. In some embodiments, a bispecific binding protein does not have one or more effector functions (e.g., “effectorless” antibodies). In some embodiments, the bispecific binding protein has no ADCC activity and/or no CDC activity. In some embodiments, the bispecific binding protein does not bind an Fc receptor and/or complement factors. In some embodiments, the bispecific binding protein has no effector function(s). In some embodiments, the constant region modifications increase or enhance ADCC and/or CDC of the bispecific binding protein. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, the bispecific binding protein comprises a variant Fc region that is engineered with substitutions at specific amino acid positions as compared to a native Fc region. In some embodiments, the bispecific binding protein described herein comprises an IgG1 heavy chain constant region that comprises one or more amino acid substitutions selected from the group consisting of K214R, L234A, L235E, G237A, D356E, and L358M, per EU numbering. In some embodiments, the IgG1 heavy chain constant region comprises one or more amino acid substitutions selected from the group consisting of K214R, L234A, L235E, G237A, A330S, P331S, D356E, and L358M, per EU numbering. In some embodiments, the IgG1 heavy chain constant region comprises one or more amino acid substitutions selected from the group consisting of K214R, C226S, C229S, and P238S, per EU numbering. In some embodiments, the IgG1 heavy chain constant region comprises one or more amino acid substitutions selected from the group consisting of K214R, D356E, and L358M, per EU numbering. In some embodiments, the IgG1 heavy chain constant region comprises one or more amino acid substitutions selected from the group consisting of S131C, K133R, G137E, G138S, Q196K, 1199T, N203D, K214R, C226S, C229S, and P238S, per EU numbering. In some embodiments, the bispecific binding protein described herein comprises an IgG2 heavy chain constant region that comprises one or more amino acid substitutions selected from the group consisting of V234A, G237A, P238S, H268A, V309L, A330S and P331S. In some embodiments, the bispecific binding protein described herein comprises an IgG4 heavy chain constant region that comprises one or more amino acid substitutions selected from the group consisting of S228P, L234A and L235A.

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis.

In some embodiments, a variant of the IgG consisting bispecific binding proteins disclosed herein can retain the ability to bind to IL17RB to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of the IgG consisting bispecific binding proteins comprises the amino acid sequence of the parent the IgG consisting bispecific binding proteins with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of the bispecific binding proteins comprises the amino acid sequence of the parent antibody or antigen-binding fragment with one or more non-conservative amino acid substitutions. In some embodiments, a variant of the bispecific binding proteins comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., IL17RB binding). In certain embodiments, the one or more conservative amino acid substitutions and/or the one or more non-conservative amino acid substitutions can enhance a biological activity of the variant, such that the biological activity of the functional variant is increased as compared to the parent binding moiety.

In some embodiments, the bispecific binding proteins described herein are chemically modified naturally or by intervention. In some embodiments, the bispecific binding proteins have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The bispecific binding proteins can comprise one or more analogs of an amino acid (including, for example, unnatural amino acids), as well as other modifications known in the art.

The bispecific binding proteins of the present disclosure can be analyzed for their physical, chemical and/or biological properties by various methods known in the art. In some embodiments, a bispecific binding protein is tested for its ability to bind to 2 different alarmins (e.g., human IL17RB and TSLP). Binding assays include, but are not limited to, surface plasmon resonance (e.g., BIAcore), ELISA, and FACS. In some embodiments, the dissociation constant of the binding agent (e.g., an antibody) for alarmin is the dissociation constant determined by surface plasmon resonance (e.g., BIAcore). In addition, antibodies can be evaluated for solubility, stability, thermostability, viscosity, expression levels, expression quality, and/or purification efficiency.

SM17 binds to human IL17RB with a dissociation constant (K_{D}) of about 1 μM. In some embodiments, the bispecific binding proteins used in the methods disclosed herein binds to IL17RB (e.g., human IL17RB) with a dissociation constant (K_{D}) of about 100 nM or less, about 40 nM or less, about 20 nM or less, about 10 nM or less, about 1 nM or less, about 0.1 nM or less, 50 μM or less, 10 μM or less, or 1 μM or less. In some embodiments, the K_{D} is about 20 nM or less. In some embodiments, the K_{D} is about 10 nM or less. In some embodiments, the K_{D} is about of about 5 nM or less. In some embodiments, the K_{D} is about 2 nM or less. In some embodiments, the K_{D} is about 1.5 nM or less. In some embodiments, the K_{D} is about 1 nM or less. In some embodiments, the K_{D} is about 0.5 nM or less. In some embodiments, the K_{D} is about 0.1 nM or less. In some embodiments, the K_{D} is about 50 μM or less. In some embodiments, the K_{D} is about 10 μM or less.

In some embodiments, a bispecific binding protein binds to IL17RB (e.g., human IL17RB) with a K_{D} within the range of 0.1-1 nM, 0.5-5 nM, 1-10 nM, 1-5 nM, 5-50 nM, 10-100 nM, or 50-500 nM. In some embodiments, the K_{D} is within the range of 0.1-1 nM. In some embodiments, the K_{D} is within the range of 0.5-5 nM. In some embodiments, the K_{D} is within the range of 1-10 nM. In some embodiments, the K_{D} is within the range of 1-5 nM. In some embodiments, the K_{D} is within the range of 5-50 nM. In some embodiments, the K_{D} is within the range of 10-100 nM. In some embodiments, the K_{D} is within the range of 50-500 nM.

In some embodiments, a bispecific binding protein used in the methods disclosed herein binds to IL17RB (e.g., human IL17RB) with an association constant (K_{A}) of about 0.8×10⁹ M⁻¹. In some embodiments, a bispecific binding protein used in the methods disclosed herein binds to IL17RB (e.g., human IL17RB) with a K_{A} of about 1×10⁶ M⁻¹ or more, about 1×10⁷ M⁻¹ or more, about 1×10⁸ M⁻¹ or more, about 5×10⁸ M⁻¹ or more, about 8×10⁸ M⁻¹ or more, about 1×10⁹ M⁻¹ or more, about 5×10⁹ M⁻¹ or more, about 1×10¹⁰ M⁻¹ or more, about 5×10¹⁰ M⁻¹ or more, about 1×10¹¹ M⁻¹ or more, about 5×10¹¹ M⁻¹ or more, or about 1×10¹² M⁻¹ or more. In some embodiments, the K_{A} is about 1×10⁷ M⁻¹ or more. In some embodiments, the K_{A} is about 5×10⁷ M⁻¹ or more. In some embodiments, the K_{A} is about 1×10⁸ M⁻¹ or more. In some embodiments, the K_{A} is about 5×10⁸ M⁻¹ or more. In some embodiments, the K_{A} is about 8×10⁸ M⁻¹ or more. In some embodiments, the K_{A} is about 1×10⁹ M⁻¹ or more. In some embodiments, the K_{A} is about 5×10⁹ M⁻¹ or more. In some embodiments, the K_{A} is about 1×10¹⁰ M⁻¹ or more.

In some embodiments, a bispecific binding protein used in the methods disclosed herein binds to IL17RB (e.g., human IL17RB) with a K_{A} within the range of about 1×10⁶-1×10⁷ M⁻¹, 5×10⁶-5×10⁷ M⁻¹, 1×10⁷-1×10⁸ M⁻¹, 5×10⁷-5×10⁸ M⁻¹, 1×10⁸-5×10⁸ M⁻¹, 1×10⁸-1×10⁹ M⁻¹, 5×10⁸-1×10⁹ M⁻¹, 5×10⁸-5×10⁹ M⁻¹, 1×10⁹-1×10¹⁰ M⁻¹, 5×10⁹-5×10¹⁰ M⁻¹, 1×10¹⁰-1×10¹¹ M⁻¹, 5×10¹⁰-5×10¹¹ M⁻¹, 1×10¹¹-1×10¹² M⁻¹, or 5×10¹¹-5×10¹² M⁻¹. In some embodiments, the K_{A} is within the range of about 1×10⁶-1×10⁷ M⁻¹. In some embodiments, the K_{A} is within the range of about 1×10⁷-1×10⁸ M⁻¹. In some embodiments, the K_{A} is within the range of about 1×10⁸-1×10⁹ M⁻¹. In some embodiments, the K_{A} is within the range of about 5×10⁸-1×10⁹ M⁻¹. In some embodiments, the K_{A} is within the range of about 5×10⁸-5×10⁹ M⁻¹. In some embodiments, the K_{A} is within the range of about 1×10⁹-1×10¹⁰ M⁻¹.

In some embodiments, a bispecific binding protein used in the methods disclosed herein dissociates from human IL17RB with a kd of 1.38×10⁻⁷ s⁻¹ or less, as determined by Octet (e.g., Bio-layer Interferometry). In some embodiments, a bispecific binding protein used in the methods disclosed herein dissociates from human IL17RB with a kd of about 5×10⁻⁴ s⁻¹ or less, about 1×10⁻⁴ s⁻¹ or less, about 2×10⁻⁵ s⁻¹ or less, about 4×10⁻⁶ s⁻¹ or less, about 8×10⁻⁷ s⁻¹ or less, about 2×10⁻⁷ s⁻¹ or less, about 4×10⁻⁸ s⁻¹ or less. In some embodiments, a bispecific binding protein used in the methods disclosed herein dissociates from human IL17RB with a kd of about 5×10⁻⁴ s⁻¹ or less. In some embodiments, the kd is about 1×10⁻⁴ s⁻¹ or less. In some embodiments, the kd is about 2×10⁻⁵ s⁻¹ or less. In some embodiments, the kd is about 4×10⁻⁶ s⁻¹ or less. In some embodiments, the kd is about 8×10⁻⁷ s⁻¹ or less. In some embodiments, the kd is about 2×10⁻⁷ s⁻¹ or less. In some embodiments, the kd is about 4×10⁻⁸ s⁻¹ or less.

Epitope mapping is a method of identifying the binding site, region, or epitope on a target protein where a bispecific binding protein binds. A variety of methods are known in the art for mapping epitopes on target proteins. These methods include but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, the bispecific binding proteins described herein are characterized by assays including, but not limited to, N-terminal sequencing, amino acid analysis, HPLC, mass spectrometry, ion exchange chromatography, and papain digestion.

In some embodiments, the bispecific binding proteins that can be used in the methods disclosed herein can bind this conformational epitope with a K_{A} of about 0.8×10⁹ M⁻¹. In some embodiments, the bispecific binding proteins used in the methods disclosed herein binds this conformational epitope with a K_{A} of about 1×10⁷ M⁻¹ or more, about 1×10⁸ M⁻¹ or more, about 5×10⁸ M⁻¹ or more, about 1×10⁹ M⁻¹ or more, about 5×10⁹ M⁻¹ or more, about 1×10¹⁰ M⁻¹ or more, about 5×10¹⁰ M⁻¹ or more, about 1×10¹¹ M⁻¹ or more, about 5×10¹¹ M⁻¹ or more, or about 1×10¹² M⁻¹ or more. In some embodiments, the K_{A} is about 1×10⁷ M⁻¹ or more. In some embodiments, the K_{A} is about 5×10⁷ M⁻¹ or more. In some embodiments, the K_{A} is about 1×10⁸ M⁻¹ or more. In some embodiments, the K_{A} is about 5×10⁸ M⁻¹ or more. In some embodiments, the K_{A} is about 8×10⁸ M⁻¹ or more. In some embodiments, the K_{A} is about 1×10⁹ M⁻¹ or more. In some embodiments, the K_{A} is about 5×10⁹ M⁻¹ or more. In some embodiments, the bispecific binding proteins used in the methods disclosed herein binds this conformational epitope with a K_{A} within the range of about 1×10⁶-1×10⁷ M⁻¹, 5×10⁶-5×10⁷ M⁻¹, 1×10⁷-1×10⁸ M⁻¹, 5×10⁷-5×10⁸ M⁻¹, 1×10⁸-5×10⁸ M⁻¹, 1×10⁸-1×10⁹ M⁻¹, 5×10⁸-1×10⁹ M⁻¹, 5×10⁸-5×10⁹ M⁻¹, 1×10⁹-1×10¹⁰ M⁻¹, 5×10⁹-5×10¹⁰ M⁻¹, 1×10¹⁰-1×10¹¹ M⁻¹, 5×10¹⁰-5×10¹¹ M⁻¹, 1×10¹¹-1×10¹² M⁻¹, or 5×10¹¹-5×10¹² M⁻¹. In some embodiments, the K_{A} is within the range of about 1×10⁶-1×10⁷ M⁻¹. In some embodiments, the K_{A} is within the range of about 1×10⁶-1×10⁷ M⁻¹. In some embodiments, the K_{A} is within the range of about 1×10⁷-1×10⁸ M⁻¹. In some embodiments, the K_{A} is within the range of about 1×10⁸-1×10⁹ M⁻¹. In some embodiments, the K_{A} is within the range of about 5×10⁸-1×10⁹ M⁻¹. In some embodiments, the K_{A} is within the range of about 5×10⁸-5×10⁹ M⁻¹. In some embodiments, the K_{A} is within the range of about 1×10⁹-1×10¹⁰ M⁻¹.

In some embodiments, provided herein is one bispecific binding protein that compete with another bispecific binding protein (e.g., human IL17RB) for binding to alarmins. Bispecific binding proteins that “compete with another antibody for binding to a target” refer to bispecific binding proteins that inhibit (partially or completely) the binding of the other bispecific binding protein to the same target. Whether competing with each other for binding to a target, i.e., whether and to what extent one bispecific binding protein inhibits the binding of another bispecific binding protein to a target, can be determined using known competition experiments, e.g., Bio-layer Interferometry Kinetic Analysis. In some embodiments, a consisting bispecific binding proteins competes with, and inhibits binding of another bispecific binding protein to alarmins (e.g., human IL17RB) by at least 50%, 60%, 70%, 80%, 90% or 100%. Competition assays can be conducted as described, for example, in Ed Harlow and David Lane, Cold Spring Harb Protoc; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, the bispecific binding proteins used in the methods disclosed herein compete with radiolabeled I¹²⁵-SM17 binding to native IL17RB on a renal carcinoma cell (e.g., TK-10 cell) with an EC₅₀ of about 0.1 μg/ml or less, about 0.2 μg/ml or less, about 0.5 μg/ml or less, about 0.8 g/ml or less, about 1 μg/ml or less, about 2 μg/ml or less, about 5.0 μg/ml or less, about 8 μg/ml or less, about 10 μg/ml or less, or about 50 μg/ml or less. In some embodiments, the EC₅₀ is about 0.1 g/ml or less. In some embodiments, the EC₅₀ is about 0.2 μg/ml or less. In some embodiments, the EC₅₀ is about 0.5 μg/ml or less. In some embodiments, the EC₅₀ is about 1 μg/ml or less. In some embodiments, the EC₅₀ is about 2 μg/ml or less. In some embodiments, the EC₅₀ is about 5 μg/ml or less. In some embodiments, the EC₅₀ is about 10 μg/ml or less. In some embodiments, the bispecific binding proteins used in the methods disclosed herein compete with radiolabeled I¹²⁵-SM17 binding to native IL17RB on a renal carcinoma cell (e.g., TK-10 cell) with an EC₅₀ within the range of about 0.1-50 μg/ml, about 0.1-10 μg/ml, about 0.1-5 μg/ml, about 0.5-10 μg/ml, about 0.5-5 μg/ml, about 1-10 g/ml or about 1-5 μg/ml. In some embodiments, the EC₅₀ is within the range of about 0.1-50 μg/ml. In some embodiments, the EC₅₀ is within the range of about 0.1-10 μg/ml. In some embodiments, the EC₅₀ is within the range of about 0.1-5 μg/ml. In some embodiments, the EC₅₀ is within the range of about 0.5-10 μg/ml. In some embodiments, the EC₅₀ is within the range of about 0.5-5 μg/ml. In some embodiments, the EC₅₀ is within the range of about 1-10 μg/ml. In some embodiments, the EC₅₀ is within the range of about 1-5 μg/ml.

In some embodiments, the bispecific binding proteins provided herein can be derivatized or linked to another functional molecule (e.g., another peptide or protein) and used in methods disclosed herein. Accordingly, in some embodiments, the antibodies and antigen-binding fragments used in methods disclosed herein include derivatized and otherwise modified forms of the human anti-IL17RB antibodies described herein, including immunoadhesion molecules. For example, the antibodies and antigen-binding fragments can be functionally linked (by chemical coupling, genetic fusion, noncovalent association or introduction of an artificial amino acid/functional group suitable for site-specific conjugation) to one or more other molecular entities, such as another antibody (e.g., a bispecific antibody or a diabody), a detectable agent, a cytotoxic agent, a pharmaceutical agent, and/or a protein or peptide that can mediate associate of the antibody or antigen-binding fragment with another molecule (such as a streptavidin core region or a polyhistidine tag).

In some embodiments, the bispecific binding proteins described herein is conjugated to a detectable substance or molecule that allows the agent to be used for diagnosis and/or detection. A detectable substance can also include, but is not limited to, enzymes, such as horseradish peroxidase, alkaline phosphatase, glucose oxidase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), 5-dimethylamine-1-napthalenesulfonyl chloride, and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions positron emitting metals; and magnetic metal ions.

The bispecific binding proteins described herein can be attached to a solid support. Such solid supports include, but are not limited to, glass, cellulose, polyacrylamide, nylon, polystyrene, polyvinyl chloride, or polypropylene. In some embodiments, an immobilized bispecific binding proteins is used in an immunoassay. In some embodiments, an immobilized the IgG consisting bispecific binding proteins is used in purification of the target antigen (e.g., human IL17RB).

### 7.3 Methods of Production

The bispecific binding proteins and antibodies thereof that can be used in methods disclosed herein, including but not limited to bispecific antibodies, antibody-alarmin receptor fusion protein, monoclonal antibodies, chimeric antibodies, human antibodies, and humanized antibodies, can be prepared by any methods disclosed herein or otherwise known in the art. Methods of antibody production are well-known in the art. See for example, in Harlow et al., ANTIBODIES: A LABORATORY MANUAL, (Cold Spring Harbor Laboratory Press, 2nd ed. 1988); Hammerling et al., in: Monoclonal Antibodies and T-Cell Hybridomas 563, 681 (Elsevier, N.Y., 1981), each of which is incorporated herein in its entirety by reference.

In some embodiments, the bispecific binding protein that can be used in methods provided herein are recombinant, namely, prepared, expressed, produced or isolated by recombinant means. In some embodiments, the bispecific binding protein disclosed herein can be prepared, for example, by introducing recombinant expression vectors into host cells, a recombinant, combinatorial human antibody library, antibodies isolated from an animal (e.g., a mouse) that is transgenic for human immunoglobulin genes [Taylor, L D et al. Nucleic Acids Research, vol. 20, 23 (1992): 6287-95] or antibodies prepared, expressed, produced, or isolated by any other means that involves splicing of human immunoglobulin gene sequences to other DNA sequences.

In some embodiments, the bispecific binding protein can be prepared by recombinant expression of immunoglobulin light and heavy chain genes in a host cell. To express a bispecific binding protein, a host cell is introduced with one or more recombinant expression vectors carrying DNA fragments encoding the immunoglobulin light and heavy chains of the bispecific binding protein such that the light and heavy chains are expressed in the host cell and, preferably, secreted into the medium in which the host cells are cultured, from which medium the bispecific binding protein can be recovered. Standard recombinant DNA methodologies are used to obtain the bispecific binding protein heavy and light chain genes, incorporate these genes into recombinant expression vectors and introduce the vectors into host cells, such as those described in Sambrook, Fritsch and Maniais (eds), MOLECULAR CLONING: A LABORATORY MANUAL, Second Edition, Cold Spring Harbor, N.Y., (1989), Ausubel et al. (eds.) CURRENT PROTOCOLS IN MOLECULAR BIOLOGY, Greene Publishing Associates, (1989) and in U.S. Pat. No. 4,816,397.

To express a recombinant bispecific binding protein, such as a SM17-related bispecific binding protein, DNA fragments encoding the light and heavy chain variable regions are first obtained. These DNAs can be obtained by amplification and modification of hybridomas for the murine antibody light and heavy chain variable sequences using the polymerase chain reaction (PCR), or by oligosynthesis based on the encoded amino acid sequence of design light and heavy chain variable sequences using standard methods known to those skilled in the art. The encoding DNA sequences can be further optimized to facilitate mammalian expression of the resultant antibody.

Once the VH and VL fragments for the murine antibody are obtained, these sequences can be mutated to encode the framework-patched version, the method of which was described in WO2020115319A1 incorporated herein in their entirety by reference.

Once DNA fragments encoding bispecific binding protein VH and VL segments are obtained (by, e.g., amplification and mutagenesis of the original murine VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, Ig4, IgA, IgE, IgM or IgD constant region, but most preferably is an IgG1 or IgG4 constant region. For an Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as an Fab light chain gene) by operatively linking the VL-encoding DNA to another DNA molecule encoding the light chain constant region, CL. The sequences of human light chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To prepare a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker [Bird, R E et al. Science, (New York, N.Y.) vol. 242, 4877 (1988): 423-6; Huston, J S et al. Proceedings of the National Academy of Sciences of the United States of America, vol. 85, 16 (1988): 5879-83; McCafferty, J et al. Nature, vol. 348, 6301 (1990): 552-4].

To express the bispecific binding proteins that can be used in the methods disclosed herein, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the bispecific binding protein heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). In some embodiments, prior to insertion of the SM17-related bispecific binding protein light or heavy chain sequences, the expression vector already carries bispecific binding protein constant regions sequences. For example, one approach to convert the SM17-related bispecific binding protein VH and VL sequences to full-length bispecific binding protein genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the bispecific binding protein chain genes, the recombinant expression vectors provided herein can carry regulatory sequences that control the expression of the bispecific binding protein chain genes in a host cell. The term “regulatory sequence” is intended to include promoters, enhancers and other expression control elements (e.g., polyadenylation signals) that control the transcription or translation of the bispecific binding protein chain genes. Such regulatory sequences are described, for example, in Goeddel; GENE EXPRESSION TECHNOLOGY: METHODS IN ENZYMOLOGY 185, Academic Press, San Diego, Calif. (1990). As those skilled in the art would appreciate, the design of the expression vector, including the selection of regulatory sequences depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from immunoglobulin heavy chain (IgH) enhancer (Gillies, S D et al. Cell, vol. 33, 3 (1983): 717-28. doi:10.1016/0092-8674(83)90014-4) metallothioneine (MT), cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. Nos. 5,665,578; 5,168,062; 4,510,245; and 4,968,615.

In addition to the bispecific binding protein chain genes and regulatory sequences, the recombinant expression vectors provided herein can carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr− host cells with methotrexate selection/amplification), Glutamate Synthase (GS) gene and the neo gene (for G418 selection).

For expression of the light and heavy chains, the expression vector encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, lipofection, protoplast fusion and the like. Although bispecific binding protein can be produced in either prokaryotic or eukaryotic host cells, expression of bispecific binding proteins in eukaryotic cells, especially mammalian host cells, is preferred because such host cells are more likely than prokaryotic cells to assemble and secrete a properly folded and immunologically active bispecific binding proteins.

Preferred mammalian host cells for expressing the recombinant bispecific binding proteins used in methods described herein include SP2/0 myeloma cells, NSO myeloma cells, COS cells, and Chinese Hamster Ovary (CHO cells) (including dfhr−CHO cells, described in Urlaub and Chasin, (1980) Proc. Natl. Acad. Sci. USA 77:4216-4200, used with a DHFR selectable marker, e.g., as described in R. J. Kaufman and P. A. Sharp (1982) J. Mol. Biol. 159:601-621). When recombinant protein-encoding expression vectors are introduced into mammalian host cells, the bispecific binding proteins are produced by culturing the host cells for a period of time sufficient to allow for expression of the bispecific binding proteins in the host cells or, more preferably, secretion of the bispecific binding protein into the culture medium in which the host cells are grown. Bispecific binding proteins can be recovered from the culture medium using standard protein purification methods.

Host cells can also be used to produce portions of intact antibodies, such as Fab fragments or scFv molecules. Expressly contemplated herein are variations of the above procedure. For example, it can be desirable to transfect a host cell with DNA encoding either the light chain or the heavy chain (but not both) of an antibody used in methods disclosed herein. Recombinant DNA technology can also be used to remove some or all of the DNA encoding either or both of the light and heavy chains that is not necessary for binding to alarmin. The molecules expressed from such truncated DNA molecules are also encompassed by the antibodies provided herein. In addition, bispecific binding protein can be produced in which one heavy and one light chain are an antibody that specifically binds human IL17RB, and the other heavy and light chain are specific for an antigen other than IL17RB by crosslinking an antibody of the invention to a second antibody by standard chemical crosslinking methods.

In some embodiments of the systems of recombinant expression of bispecific binding proteins thereof that can be used in the methods disclosed herein, a recombinant expression vector encoding both the heavy chain and the antibody light chain is introduced into SP2/0 cells by electroporation. In some embodiments of the expression systems, a recombinant expression vector encoding both the heavy chain and the antibody light chain is introduced into CHO cells by standard techniques such as lipofection.

Within the recombinant expression vector, the heavy and light chain genes are each operatively linked to murine or human Immunoglobulin heavy chain (IgH), CMV enhancer, metallothioneine or AdMLP promoter regulatory elements to drive high levels of transcription of the genes. The recombinant expression vector also carries a DHFR gene, which allows for selection of SP2/0 cells that have been transfected with the vector using methotrexate selection/amplification. Alternatively, the recombinant expression vector containing the heavy and light chain genes operatively linked to murine or human IgH, CMV enhancer/AdMLP/metallothioneine promoter regulatory elements and a DHFR gene can be used to transfect SP2/0 or CHO cells that are dhfr-. SP2/0 or CHO cells transfected with the vector can be selected and the level of gene expression in the vector amplified by increasing the levels of methotrexate in the culture. The selected transformant host cells are cultured to allow for expression of the heavy and light chains and intact bispecific binding protein is recovered from the culture medium. Standard molecular biology techniques are used to prepare the recombinant expression vector, transfect the host cells, select for transformants, culture the host cells and recover the bispecific binding proteins from the culture medium.

### 7.4 Pharmaceutical Compositions

Provided herein are also pharmaceutical compositions comprising the bispecific binding proteins that can be used in methods disclosed herein. In some embodiments, the pharmaceutical composition comprises a therapeutically effective amount of the bispecific binding proteins disclosed herein and a pharmaceutically acceptable carrier. In some embodiments, the pharmaceutical compositions are useful in treating AD and asthma. In some embodiments, the pharmaceutical compositions are useful in inhibiting AD and asthma progression in a subject (e.g., a human patient).

The amount of therapeutic bispecific binding proteins which can be combined with a carrier material in the pharmaceutical compositions disclosed herein can vary. In some embodiments, the amount of bispecific binding proteins present in the pharmaceutical compositions is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions provided herein comprise the bispecific binding proteins provided herein, e.g., SM17-related bispecific binding proteins. The bispecific binding proteins can be present at various concentrations. In some embodiments, the pharmaceutical compositions provided herein comprise soluble bispecific binding proteins provided herein at 1-1000 mg/ml. In some embodiments, the pharmaceutical compositions comprise soluble bispecific binding proteins provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions provided herein comprise the bispecific binding proteins provided herein at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml. Dosages can be readily adjusted by those skilled in the art; for example, a decrease in purity requires an increase in dosage.

The pharmaceutical compositions provided herein can be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants. In some embodiments, pharmaceutical compositions provided herein are in the form of injectable or infusible solutions. In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. The composition can be formulated as a solution, microemulsion, liposome, or other ordered structure suitable to high drug concentration.

In some embodiments, the pharmaceutical compositions disclosed herein are freeze-dried, to which the physician or the patient adds solvents and/or diluents prior to use.

The pharmaceutical compositions provided herein can comprise a pharmaceutically acceptable carrier. Pharmaceutically acceptable carriers include any and all solvents, dispersion media, coatings, antibacterial and antifungal agents, isotonic and absorption delaying agents, and the like that are physiologically compatible. Examples include one or more of water, saline, phosphate buffered saline, dextrose, glycerol, ethanol and the like, as well as combinations thereof. In some embodiments, the pharmaceutical acceptable carriers include isotonic agents, for example, sugars, polyalcohols such as mannitol, sorbitol, or sodium chloride in the composition.

In some embodiments, the pharmaceutical acceptable carriers further comprise minor amounts of auxiliary substances such as wetting or emulsifying agents, preservatives or buffers, which enhance the shelf life or effectiveness of the antibody or antigen-binding fragment. In some embodiments, the carrier is suitable for intravenous, intramuscular, subcutaneous, parenteral, spinal or epidermal administration (e.g., by injection or infusion). Depending on the route of administration, the active ingredient (i.e., the bispecific binding proteins), can be coated in a material to protect the active ingredient from the action of acids and other natural conditions that can inactivate the active ingredient.

Provided herein are also kits for preparation of pharmaceutical compositions having the bispecific binding proteins disclosed herein, e.g., SM17-related bispecific binding proteins. In some embodiments, the kit comprises the bispecific binding proteins disclosed herein and a pharmaceutically acceptable carrier in one or more containers. In another embodiment, the kits can comprise the binding proteins disclosed herein for administration to a subject. In specific embodiments, the kits comprise instructions regarding the preparation and/or administration of the bispecific binding proteins.

In some embodiments, the pharmaceutical composition or formulation disclosed herein comprises: (a) the bispecific binding proteins disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C. In some embodiments, provided herein are also pharmaceutical compositions or formulations that improve the stability of the bispecific binding proteins to allow for their long-term storage. The pharmaceutical compositions disclosed herein can further comprise one or more of a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: THE SCIENCE AND PRACTICE OF PHARMACY, 19th edition, 1995.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, raffinose, or maltose, polyols such as glycerol, mannitol, sorbitol, cyclodextrins or dextrans of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutical compositions or formulations typically must be sterile and stable under the conditions of manufacture and storage. Pharmaceutically acceptable carriers include sterile aqueous solutions or dispersions and sterile powders for the extemporaneous preparation of sterile injectable solutions or dispersion. Sterile injectable solutions can be prepared by incorporating the therapeutic antibody or antigen-binding fragment in the required amount in an appropriate solvent with one or a combination of ingredients enumerated above, as required, followed by filtered sterilization. The use of such media and agents for pharmaceutically active substances is known in the art. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., SUSTAINED AND CONTROLLED RELEASE DRUG DELIVERY SYSTEMS, J. R. Robinson, ed., Marcel Dekker, Inc., New York, 1978.

In some embodiments, the bispecific binding proteins described herein can be formulated to ensure proper distribution in vivo. For example, the blood-brain barrier (BBB) excludes many highly hydrophilic compounds. To facilitate the therapeutic antibodies described herein to cross the BBB, they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin [see, e.g., U.S. Pat. No. 5,416,016 to Low et al] mannosides [Umezawa, F, and Y Eto. Biochemical And Biophysical Research Communications vol. 153, 3 (1988): 1038-44.]; antibodies [Bloemen, P G et al. FEBS letters vol. 357, 2 (1995): 140-4.] [Owais, M et al. Antimicrobial Agents And Chemotherapy vol. 39, 1 (1995): 180-4.]; surfactant protein A receptor [Briscoe, P et al. The American Journal Of Physiology vol. 268, 3 Pt 1 (1995): L374-80]; p120 [Schreier, H et al. The Journal Of Biological Chemistry vol. 269, 12 (1994): 9090-8][Keinanen, K, and M L Laukkanen. FEBS letters vol. 346, 1 (1994): 123-6] [Killion, J J, and I J Fidler. ImmunoMethods vol. 4, 3 (1994): 273-9].

### 7.5 Methods of Treatment

As described in sections above, provided herein are medical uses of bispecific binding proteins (e.g., SM17-related bispecific binding proteins) in treating allergic diseases or disorders. Any bispecific binding protein disclosed herein can be used in the methods disclosed herein. In some embodiments, used in methods disclosed herein are recombinant anti-IL17RB/anti-TSLP bispecific antibodies. In some embodiments, used in methods disclosed herein are recombinant anti-IL17RB/anti-IL33 bispecific antibodies. In some embodiments, used in methods disclosed herein are recombinant anti-IL17RB/TSLP receptor bispecific binding proteins. In some embodiments, used in methods disclosed herein are recombinant anti-IL17RB/IL33 receptor bispecific binding proteins.

In some embodiments, provided herein are methods of reducing pulmonary ILC2 proliferation in a subject in need thereof. In some embodiments, provided herein are also methods of treating a disease or disorder related to allergy in a subject in need thereof. In some embodiments, provided herein are also methods of reducing eosinophilic inflammation in a subject in need thereof. In some embodiments, provided herein are also methods of treating a disease or disorder related with neutrophilic inflammation in a subject in need thereof. In some embodiments, the methods provided herein of treating a disease or disorder by reducing OCS daily dose in a subject in need thereof by at least 20%, at least 50%, or at least over 75%. In some embodiments, the subject is a human.

The methods of reducing pulmonary ILC2 proliferation, treating a disease or disorder related with neutrophilic inflammation, treating a disease or disorder related with eosinophilic inflammation, and treating a disease or disorder by reducing OCS daily dose comprise administering to the subject a therapeutically effective amount of the bispecific binding protein that specifically binds (a) IL17RB and TSLP and/or (b) IL17RB and IL-33.

Subjects suitable for the present methods include human patients in whom the blockade of alarmins' activity would be desirable. In some embodiments, the subjects to be treated with the methods disclosed herein are diagnosed with disease or disorder related to allergy, which can be clinical or pre-clinical asthma, AD, fibrotic disease, inflammatory bowel disease (IBD), Crohn's disease, ulcerative colitis, chronic obstructive pulmonary disease, chronic sinusitis, chronic rhinosinusitis with nasal polyps. In some embodiments, the subject can be a mammal. In some embodiments, the subject is a human. In some embodiments, the subject to be treated with the methods disclosed herein have been with the OCS. In some embodiments, the subject has not been previously treated.

The bispecific binding proteins (e.g., SM17 related bispecific binding proteins) or pharmaceutical compositions provided herein can be administered to a subject by any methods known in the art, including, but not limited to, intravenous administration, subcutaneous administration, intramuscular administration, intracranial administration, intrathecal administration, intraventricular administration, intraperitoneal administration, spinal administration, intranasal administration, intrapleural administration, topical administration, or intradermal administration.

In some embodiments, the bispecific binding proteins (e.g., SM17 related bispecific binding proteins) or pharmaceutical compositions provided herein can be administered to a subject using parenteral administration. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, the bispecific binding proteins are administered by intravenous infusion or injection. In some embodiments, the bispecific binding proteins are administered by intramuscular injection. In some embodiments, the bispecific binding proteins are administered by subcutaneous injection.

The bispecific binding proteins (e.g., SM17 related bispecific binding proteins) or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the bispecific binding proteins disclosed herein can be orally administered, for example, with an inert diluent or an assimilable edible carrier. The therapeutic bispecific binding proteins can also be enclosed in a hard- or soft-shell gelatin capsule, compressed into tablets, or incorporated directly into the subject's diet. For oral therapeutic administration, the bispecific binding proteins can be incorporated with excipients and used in the form of ingestible tablets, buccal tablets, troches, capsules, elixirs, suspensions, syrups, wafers, and the like.

The methods provided herein comprise administering a therapeutically effective amount of the bispecific binding proteins (e.g., SM17 related bispecific binding proteins) described herein. Actual dosage levels of the therapeutic antibodies can be varied so as to obtain an amount which is effective to achieve the desired therapeutic response for a particular patient, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Generally, the dosage can range from, e.g., about 0.1 to 100 mg/kg of the host body weight for a single dose. In some embodiments, the bispecific binding proteins (e.g., SM17 related bispecific binding proteins) is administered at about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg per. In some embodiments, the bispecific binding proteins is administered at about 1 mg/kg. In some embodiments, the bispecific binding proteins is administered at about 5 mg/kg. In some embodiments, the bispecific binding proteins is administered at about 10 mg/kg. In some embodiments, the bispecific binding proteins is administered at about 20 mg/kg. In some embodiments, the bispecific binding proteins is administered at about 40 mg/kg. In some embodiments, the bispecific binding proteins is administered at about 60 mg/kg. In some embodiments, the bispecific binding proteins is administered at about 100 mg/kg.

In some embodiments, the bispecific binding proteins (e.g., SM17 related bispecific binding protein) is administered at a dose within a range of about 1 to 5 mg/kg, about 1 to 10 mg/kg, about 1 to 20 mg/kg, about 1 to 50 mg/kg, about 1 to 100 mg/kg, about 5 to 10 mg/kg, about 5 to 20 mg/kg, about 5 to 50 mg/kg, about 5 to 100 mg/kg, about 10 to 50 mg/kg, or about 10 to 100 mg/kg. In some embodiments, the bispecific binding proteins is administered at a dose within a range of about 1 to 5 mg/kg. In some embodiments, the bispecific binding proteins is administered at a dose within a range of about 1 to 10 mg/kg. In some embodiments, the bispecific binding proteins is administered at a dose within a range of about 1 to 50 mg/kg. In some embodiments, the bispecific binding proteins is administered at a dose within a range of about 10 to 50 mg/kg. In some embodiments, the bispecific binding proteins is administered at a dose within a range of about 10 to 100 mg/kg.

In some embodiments, methods provided herein comprise administering the bispecific binding proteins (e.g., SM17 related bispecific binding protein) at a dose of about 10-2000 mg. In some embodiments, the dose is about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg about 1600 mg, about 1700 mg, about 1800 mg, about 1900 mg, or about 2000 mg. In some embodiments, the antibody is administered at a dose of 100 mg. In some embodiments, the antibody is administered at a dose of 300 mg. In some embodiments, the antibody is administered at a dose of 600 mg. In some embodiments, the antibody is administered at a dose of 900 mg. In some embodiments, the antibody is administered at a dose of 1200 mg.

In some embodiments, methods provided herein comprise administering the IgG consisting bispecific binding proteins (e.g., SM17 related bispecific binding protein) at a dose within a range of about 10-50 mg, 10-100 mg, 10-200 mg, 100-300 mg, 100-500 mg, 300-600 mg, 300-900 mg, 300-1200 mg, 600-1200 mg, 600-1800 mg, or 1000-2000 mg. In some embodiments, the antibody is administered at a dose within the range of 100-500 mg. In some embodiments, the antibody is administered at a dose within the range of 300-600 mg. In some embodiments, the antibody is administered at a dose within the range of 300-900 mg. In some embodiments, the antibody is administered at a dose within the range of 600-1200 mg.

During treatment, it is common to start with a lower dose, which is later ramped up to a target dose. For illustrative purpose, in some embodiments, the methods provided herein comprise administering the bispecific binding proteins at a dose of about 100 mg, which is gradually ramped up to a target dose of about 600 mg.

Subjects can be administered at such doses daily, on alternative days, weekly, biweekly, monthly, or according to any other schedule determined by empirical analysis. An exemplary treatment entails administration in multiple dosages over a prolonged period, for example, of at least six months. In some embodiments, the methods provided herein comprise weekly administering of the bispecific binding proteins. In some embodiments, the methods comprise biweekly administration. In some embodiments, the methods comprise monthly administration. In some embodiments, the bispecific binding proteins (e.g., SM17 related bispecific binding protein) is subcutaneously administered weekly, biweekly, or monthly. In some embodiments, the IgG consisting bispecific binding proteins (e.g., SM17 related bispecific binding protein) is intravenously administered weekly, biweekly, or monthly.

The bispecific binding proteins (e.g., SM17 related bispecific binding protein) can be administered up to 3 months, 6 months, 9 months, 12 months, 18 months, 24 months, 30 months, or 36 months, as necessary and appropriate. In some embodiments, the treatment lasts at least 3 months. In some embodiments, the treatment lasts at least 6 months. In some embodiments, the treatment lasts at least 12 months. In some embodiments, the treatment lasts at least 24 months.

All permutations and combinations of the various embodiments of, e.g., round of administration, dosage, treatment frequency, and length of treatment are expressly contemplated herein and can be adopted in the therapeutic methods disclosed herein.

For illustrative purposes, the following treatment regimen can be adopted in the methods disclosed herein that comprise administering of an anti-IL17RB antibody or antigen binding fragment that is either disclosed herein (e.g., SM17 related bispecific binding protein) or identified in methods disclosed herein:

In some embodiments, the therapeutic antibody is administered intravenously or subcutaneously at a dose of about 10 mg/kg every 4 weeks and at least 21 days apart. In some embodiments, the following titration schedule is included: Infusions 1-2: 1 mg/kg IV; Infusions 3-4: 3 mg/kg IV; Infusions 5-6: 6 mg/kg IV; Infusion 7 and beyond: 10 mg/kg IV.

In some embodiments, the therapeutic antibody is administered intravenously or subcutaneously at a single dose of 10, 20, or 40 mg/kg, the second of 10 mg/kg every other week for 24 weeks, and the third of 10 or 20 mg/kg every month for 16 months.

In some embodiments, the therapeutic antibody is administered intravenously or subcutaneously at a dose of about 250 mg weekly, or 500 mg biweekly for up to 2 years. In some embodiments, the treatment starts with monthly shots of about 120 mg.

Dosage regimens can be adjusted to provide the optimum desired response (e.g., a therapeutic or prophylactic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of therapeutic antibody calculated to produce the desired therapeutic effect in association with the required pharmaceutical carrier. It is to be noted that proper dosing varies with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In the treatment of a disease or disorder related to allergy, sometimes the disease or disorder can be cured with methods provided herein, but any clinical improvement constitutes a benefit. In some embodiments, the methods provided herein reduce daily OCS usage by an average of about 2.5 mg/day, about 5 mg/day, about 10 mg/day, about 20 mg/day, about 40 mg/day, about 95 CL. In some embodiments, the methods provided herein reduce OCS dose daily usage by an average of 25 percentage. In some embodiments, the methods provided herein reduce OCS dose daily usage by an average of 50 percentage. In some embodiments, the methods provided herein reduce OCS dose daily usage by an average of 75 percentage. In some embodiments, the methods provided herein reduce OCS dose daily usage by an average of 100 percentage. In some embodiments, the methods provided herein reduce OCS dose daily usage by an average of 25-50 percentage. In some embodiments, the methods provided herein reduce OCS dose daily usage by an average of 50-75 percentage. In some embodiments, the methods provided herein reduce OCS dose daily usage by an average of 75-100 percentage.

In some embodiments, the methods provided herein reduce ILC2 proliferation in lung. In some embodiments, methods provided herein reduce ILC2 migration to lung. In some embodiments, methods provided herein reduce IL-5 levels in bronchial alveolar liquid. In some embodiments, methods provided herein reduce IL-13 levels in bronchial alveolar liquid. In some embodiments, methods provided herein reduce pulmonary eosinophilic inflammation. In some embodiments, methods provided herein reduce pulmonary neutrophilic inflammation. In some embodiments, methods provided herein reduce annual asthma exacerbation rate. In some embodiments, methods provided herein reduce the fraction of exhaled nitric oxide. In some embodiments, methods provided herein reduce the blood eosinophil counts. In some embodiments, methods provided herein reduce transepidermal water loss.

In some embodiments, methods provided herein prevent the onset of AD, or delay or halt the progression AD. In some embodiments, methods provided herein ameliorate the symptoms of AD. In some embodiments, methods provided herein prevent the onset of asthma, or delay or halt the progression asthma. In some embodiments, methods provided herein ameliorate the symptoms of asthma.

The bispecific binding proteins disclosed herein can be administered by a variety of methods known in the art. As appreciated by those skilled in the art, the route and/or mode of administration varies depending upon the desired results. In some embodiments, the bispecific binding proteins can be prepared with a carrier that protects it against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesteers, and polylactic acid. Many methods for the preparation of such formulations are patented or generally known to those skilled in the art. See, e.g., SUSTAINED AND CONTROLLED RELEASE DRUG DELIVERY SYSTEMS, J. R. Robinson ed., Marcel Dekker, Inc., New York, 1978. In therapeutic applications, a relatively high dosage at relatively short intervals is sometimes required until progression of the disease is reduced or terminated, and until the patient shows partial or complete amelioration of symptoms of disease.

Combination therapy using agents with different mechanisms of action can result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that drug-resistance would develop. In some embodiments, the additional therapy results in an increase in the therapeutic index of the bispecific binding proteins, or pharmaceutical compositions described herein. In some embodiments, the additional therapy results in a decrease in the toxicity and/or side effects of the bispecific binding proteins or pharmaceutical compositions described herein. In some embodiments, the bispecific binding proteins, or pharmaceutical compositions described herein can be administered in combination with an additional therapy.

In some embodiments, the second therapeutic agent is corticosteroid, DNA methyltransferase (DNMT) inhibitors, an anti-IL4 antibody, an anti-IL5 antibody, an anti-IL4Ra antibody, an anti-IL13 antibody, and anti-IgE antibody, an anti-IL17A antibody, anti-IL12/IL23 antibody, anti-IL23 antibody, anti-IL17RA antibody, tyrosine kinase inhibitors. In some embodiments, the second therapeutic agent can be a second antibody that suppresses the release of pro-inflammatory cytokines.

The second therapeutic agent can be administered prior to, concurrently with, or subsequent to administration of the bispecific binding proteins or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

### 7.6 Experimental Examples

### Example 1: SM17 Binds to Exogenous Murine, Monkey, and Human IL-17RB Protein

A standard enzyme-linked immunosorbent assay (ELISA) method was used to determine the species specificity of SM17 against IL-17RB proteins. Briefly, IL-17RB proteins from human, mouse, cynomolgus or rhesus monkey (R&D systems, Minneapolis, MN) were diluted to 2.5 μg/ml in PBS and 50 μl of IL-17RB proteins were added to each well of ELISA strip. The strips were sealed with parafilm and incubated at 4° C. overnight for coating.

On the next day, strips were washed thrice with washing buffer (0.05% Tween 20 in PBS) and blocked with 100 μl per well with blocking buffer (3% BSA in PBS) for 2 hours RT before serially diluted SM17 (at a final concentration of 10, 2, 0.4, 0.08, 0.016, 0.0032, and 0.00064 μg/ml, respectively) was added. After an incubation period of 2 hrs at RT, the amount of bound SM17 were revealed by the addition of peroxidase conjugated goat anti-human F(ab′)2 specific antibody (Jackson ImmunoResearch) and TMB substrate (Sigma-Aldrich, St. Louis, MO) at OD 450 nM following standard ELISA protocol known to those skilled in the art.

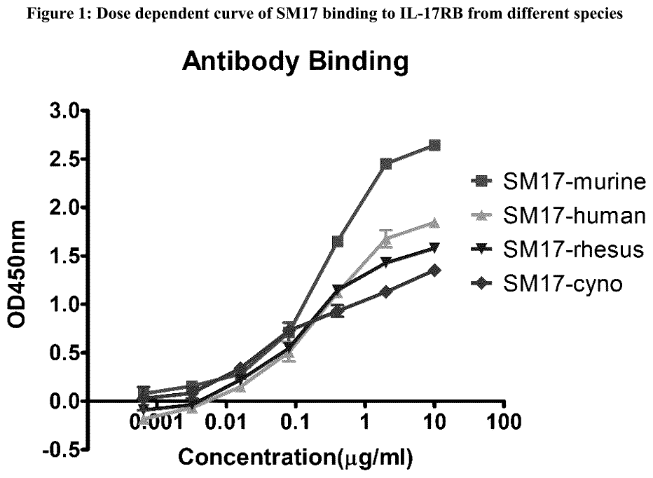

Results indicate that SM17 binds to IL-17RB proteins from murine, human, cynomolgus monkey and rhesus monkey with comparable affinity in a dose-response manner (See Table 1 and FIG. 1).

TABLE 1
Optic density of antigen binding at 450 nm to IL-17RB from different species

| SM17 | OD 450 nm Absorption | | | | | | | |
|---|---|---|---|---|---|---|---|---|
| (μg/mL) | murine IL17RB | | human IL17RB | | rhesus IL17RB | | cynomolgus IL17RB | |
| 10 | 2.67 | 2.61 | 1.82 | 1.88 | 1.57 | 1.59 | 1.33 | 1.37 |
| 2 | 2.46 | 2.44 | 1.74 | 1.61 | 1.40 | 1.45 | 1.13 | 1.12 |
| 0.4 | 1.68 | 1.61 | 1.10 | 1.14 | 1.12 | 1.17 | 0.89 | 0.97 |
| 0.08 | 0.78 | 0.64 | 0.58 | 0.44 | 0.51 | 0.58 | 0.72 | 0.74 |
| 0.016 | 0.25 | 0.31 | 0.17 | 0.13 | 0.27 | 0.17 | 0.34 | 0.35 |
| 0.0032 | 0.12 | 0.19 | −0.07 | −0.06 | −0.09 | 0.01 | 0.04 | 0.13 |
| 0.00064 | 0.04 | 0.11 | −0.16 | −0.21 | −0.10 | −0.09 | −0.06 | 0.11 |

### Example 2: SM17 does not Cross-React with Other IL17 Receptor Subunits

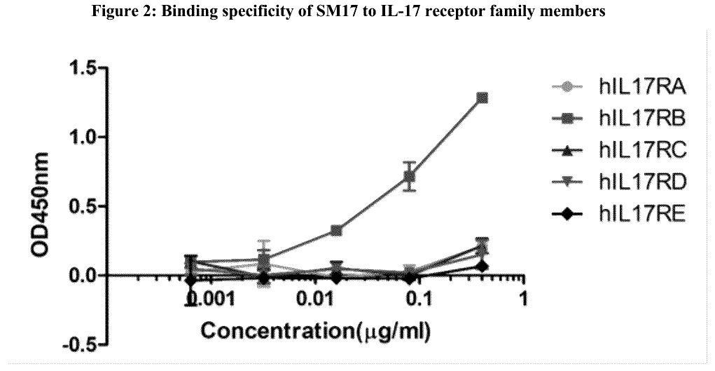

A standard ELISA method was used to determine the specificity of SM17 against other human IL17 receptor subunits. Similarly, serially diluted SM17 at different concentrations (final concentrations at 0.4, 0.08, 0.016, 0.0032 and 0.00064 μg/m, respectively) was added to the wells of ELISA plates coated either with human IL-17 receptor subunits A, C, D and E (IL17RA, IL17RC, IL17RD, and IL17RE) (R&D systems). No obvious bindings of SM17 to other known human IL-17 receptor subunits (IL17RA, IL17RC, IL17RD, and IL17RE) was detected (See Table 2 and FIG. 2).

TABLE 2
Optic density of antigen binding at 450 nm to IL-17 receptor family members

| | OD 450 nm Absorption | | | | | | | | | |
|---|---|---|---|---|---|---|---|---|---|---|
| | hIL17RA | | hIL17RB | | hIL17RC | | hIL 17RD | | hIL17RE | |
| SM17 | Well | Well | Well | Well | Well | Well | Well | Well | Well | Well |
| (μg/mL) | 1 | 2 | 1 | 2 | 1 | 2 | 1 | 2 | 1 | 2 |
| 0.4 | 0.21 | 0.22 | 1.26 | 1.31 | 0.25 | 0.18 | 0.22 | 0.08 | 0.08 | 0.05 |
| 0.08 | 0.00 | 0.06 | 0.79 | 0.64 | 0.02 | −0.02 | 0.03 | 0.01 | −0.04 | 0.00 |
| 0.016 | 0.01 | −0.04 | 0.35 | 0.30 | 0.09 | 0.02 | 0.07 | 0.02 | 0.00 | −0.04 |
| 0.0032 | 0.20 | −0.03 | 0.16 | 0.07 | 0.03 | −0.04 | 0.04 | −0.04 | −0.01 | −0.02 |
| 0.00064 | 0.00 | 0.05 | 0.13 | 0.07 | 0.13 | 0.08 | 0.08 | 0.02 | 0.09 | −0.16 |

### Example 3: SM17 Binds to Native IL-17RB from Human and Rhesus Monkey that are Expressed on the Surface of HEK293 Cells

HEK293 cells (ATCC, Manassas, VA) transfected either with full-length human or rhesus monkey IL-17RB (SinoBiological, Beijing, China) were examined for binding to SM17 following standard protocol for flow cytometry. Wild-type HEK293 stained with IgG4 isotype control (Sino Biological) was used as the negative control and for gating purpose. Briefly, HEK293 cells at a density of 3×10⁵ cells were seeded into each well of a 6-well plate. On the next day, human or rhesus Il-17RB full length cDNA were first cloned into pCMV3-untagged expression plasmid through standard molecular cloning technique, and then expression plasmids for human or rhesus IL-17RB were transfected into HEK293 cells by lipofection (Lipofectamine 3000 reagent; Thermo Fisher Scientific). Transfected cells were trypsinized, harvested, and fixed with 4% paraformaldehyde (5 minutes). SM17, or human IgG4 control at 1 μg/ml in washing buffer (3% BSA in PBS) were added to the resuspend cells for incubation at RT for 30 min before Alexa Fluor® 647-conjugated goat anti-human IgG specific antibody (1:2000 dilution in washing buffer) (Jackson ImmunoResearch) was added for flow cytometry analysis using the BD FACSVerse cell analyzer (Becton Dickenson, Franklin Lakes, NJ).

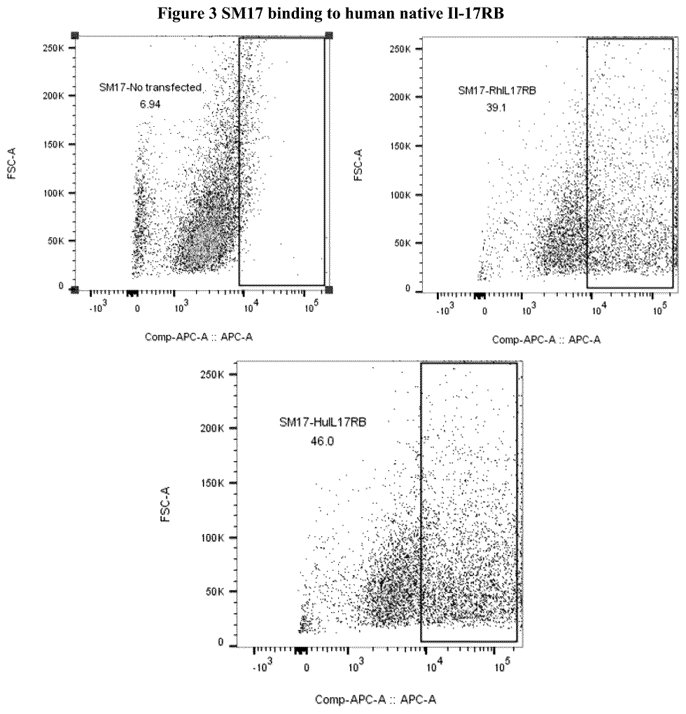

Enhanced binding of SM17 to HEK293 transfected with human IL17RB (hIL17RB-HEK293: 46%), and rhesus monkey IL17RB (RhIL17RB-HEK293: 39%) over that of the wild type HEK293 (˜7%) indicated that SM17 binds to the native human or rhesus monkey IL17RB expressed on HEK293 cells (FIG. 3).

### Example 4: SM17 Inhibits IL-5 Releases from Human PBMC Co-Cultured with IL-2 & IL-25

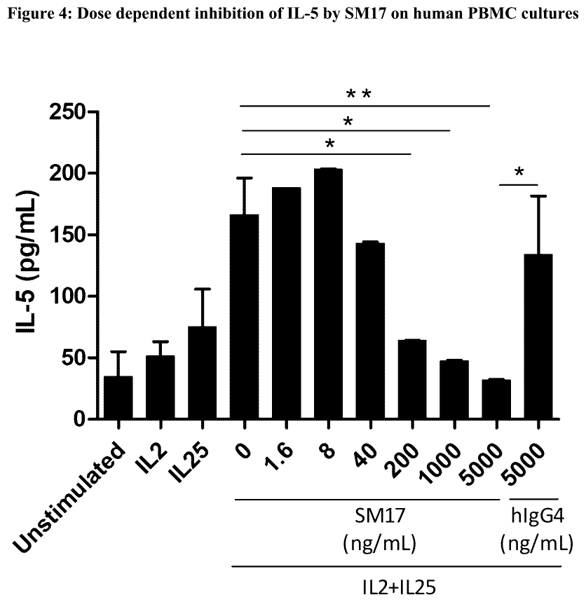

PBMC (Ixcells, San Diego, CA) co-cultured with IL-2 and IL-25 (PeproTech, Cranbury, NJ) can lead to the release of IL-5, a cytokine known to exacerbate the conditions of asthma. Accordingly, the inhibitory effect of SM17 on IL-5 release from IL-2/IL-25-treated human PBMC was evaluated. Briefly, cryopreserved human PBMC were thawed and cultured in RPMI-1640 medium supplemented with 10% Fetal Bovine Serum (Thermo Fisher Scientific). Serially diluted SM17 at concentrations ranging from 0.16 ng/mL to 5 mg/mL were added to 4×10⁵ of human PBMC in the presence of 10 unit/mL of IL-2 and 10 μg/mL of IL-25. Non-specific human IgG4 (SinoBiological) was used as the control antibody. Treated cells were incubated for 72 hours at 37° C., and the level of IL-5 release in the culture supernatant was measured by standard ELISA assay (R&D systems). Results indicated that SM17 suppressed the release of IL-5 from human PBMC induced with IL-2 and IL-25 in a dose dependent manner (FIG. 4). Results are presented as means±SEM. One Way ANOVA, Dunnett's Multiple Comparison Test, *: P<0.05; **: P<0.01.

### Example 5: Inhibition of IL-8 Release by SM17 on Renal Carcinoma Cell Line TK-10

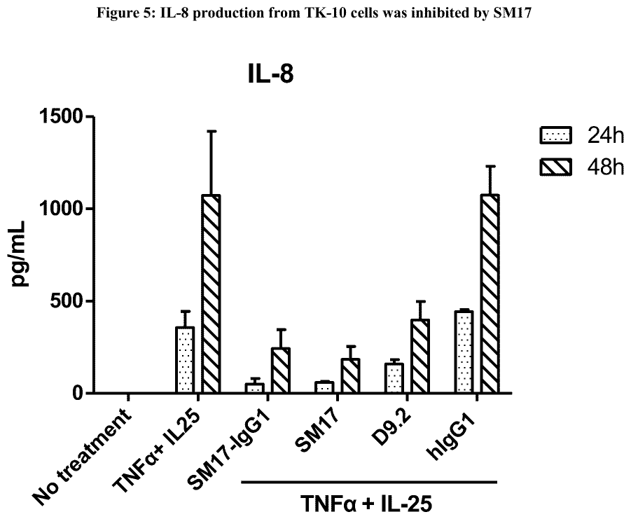

Human renal carcinoma cell line TK-10 (NCI-60, NIH, Bethesda, MD) treated with human IL-25 (PeproTech) and TNFa (R&D systems) can lead to the release of IL-8, also known as neutrophil chemotactic factor, an important mediator of the immune reaction in the innate immune system response. Accordingly, the inhibitory effects of SM17 and its murine counterpart (D9.2) on IL-8 release from IL-25/TNFa-treated TK-10 cells was evaluated. Briefly, SM17 (human IgG4 isotype, SinoMab BioScience Limited, Hong Kong, China), SM17-IgG1 (human IgG1 isotype, SinoMab BioScience Limited) and its murine counterpart (D9.2, SinoMab BioScience Limited) at a concentration of 1 mg/mL were added to 2×10⁴ of TK-10 cells in the presence of 100 ng/mL of IL-25 and 10 ng/mL of TNFa. Non-specific human IgG1 (SM03, anti-CD22 chimeric antibody, SinoMab BioScience Limited) was used as the control antibody. Treated cells were cultured in OptiMEM (Thermo Fisher Scientific) for 24 or 48 hours at 37° C., and the level of IL-8 release in the culture supernatant was measured by standard ELISA assay using a commercial kit (R&D systems). Results indicated that at 1 mg/mL, either SM17, SM17-IgG1 or D9.2 can efficiently suppress the release of IL-8 by IL-25/TNFa-induced TK-10 cells at both time points (FIG. 5).

### Example 6: Determination of Antigen (IL17RB) Binding Kinetics of SM17

Bio-Layer Interferometry analysis (Octet ReD96 system, Sartorius) was employed to determine the binding affinity of SM17 against human and cynomolgus monkey IL17RB protein (R&D systems). Briefly, SM17 (20 μg/mL) was immobilized on biosensors via interactions with anti-human Fab CH1; serially diluted Cyno-IL17RB and Human-IL17RB (at a concentration of 158.7 nM, 79.4 nM and 39.7 nM, respectively) were subsequently added following standard operation protocols of the Octet ReD96 system to plot out the association and dissociation curve. Irrelevant antibody (SM03, anti-human CD22 chimeric IgG1 antibody, SinoMab BioScience Limited) was used as the control reference. Kₐ, K_{dis} and K_{D} values of the respective antibodies are summarized in Table 3 below. Of note, the K_{D} is within picomolar-single digit nanomolar range.

TABLE 3
Binding Kinetics of SM17 to human and cynomolgus IL-17RB

| | | Kₐ | K_{dis} | K_{D} | Range |
|---|---|---|---|---|---|
| IL17RB | Trial | (1/Ms) | (1/s) | (nM) | (nM) |
| Human | 1 | 2.22 × 10⁵ | 4.30 × 10⁻⁵ | 0.194 | 0.194-0.546 |
| | 2 | 1.83 × 10⁵ | 9.98 × 10⁻⁵ | 0.546 | |
| Cynomolgus | 1 | 3.21 × 10⁴ | 2.73 × 10⁻⁵ | 0.852 | 0.852-5.11 |
| | 2 | 4.21 × 10⁴ | 2.15 × 10⁻⁴ | 5.11 | |

### Example 7: Therapeutic Effects of SM17 on Ovalbumin Induced Asthma on Mice

Female BALB/c mice were induced with ovalbumin to elicit asthma like symptoms on mice. Briefly, mice were each sensitized with 10 mg of ovalbumin (OVA) emulsified in 1 mg of aluminum hydroxide in a total volume of 200 mL via intraperitoneal (IP) injections on Days 0 and 12. Mice were divided into 6 groups with 8 mice per group. Sensitized mice were exposed to aerosolized 5% OVA in sterile water, 20 minutes/day for six consecutive days (Days 19, 20, 21, 22, 23 and 24). Naïve mice inhaled the atomized water, 20 minutes/day, for six consecutive days. Mice were then i.v. injected with different concentrations of SM17, PBS (control), or dexamethasone (Dex) 4 hours before aerosol challenge, once per day (Day 19 to 24).

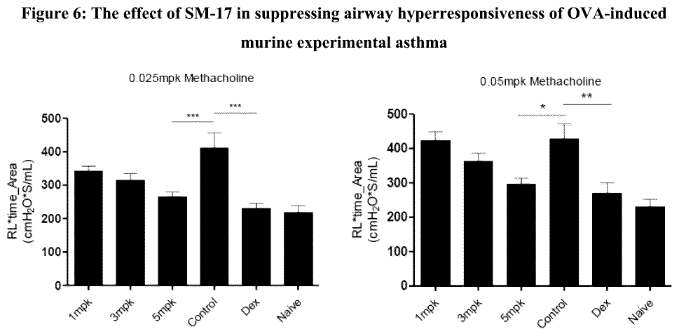

On day 25 (24 hours after the last OVA challenge), mice were anesthetized with Telazol® (20-40 mg/kg i.p.) and connected to a computer-controlled ventilator via the tracheal cannula. The time of expiration/inspiration and the respiratory rate were preset at 1.5:1 and 90/min, respectively. After a steady baseline was established, the resistance of the lung (RL) was recorded to evaluate the reaction of mice to a methacholine chloride gradient (0.025 and 0.05 mg/kg body weight); the methacholine chloride was injected into the vena jugulars externa at 5-min intervals via a fine needle. Mice treated with 5 mg/kg SM17 or 1 mg/kg Dex prevented airway hyperresponsiveness. The administration of 1 mg/kg or 3 mg/kg SM17 before each OVA aerosolization resulted in a mild but not significant abrogation in AHR after challenge with methacholine. As the results shown in FIG. 6 demonstrate, the therapeutic effects of SM17 are dose-dependent. BALF from mice sacrificed on Day 25 was retrieved, and inflammatory cell number and the concentrations of inflammatory cytokine from BALF were determined.

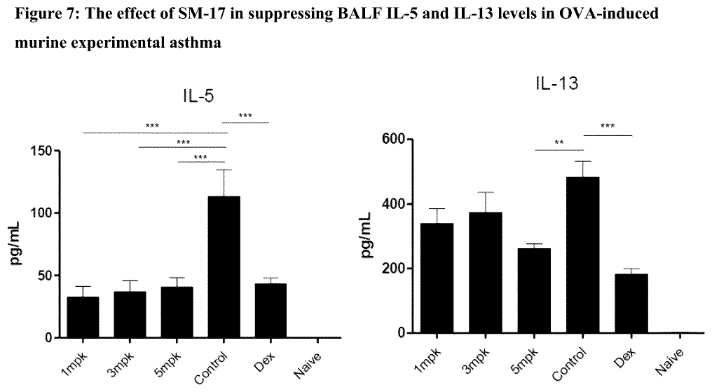

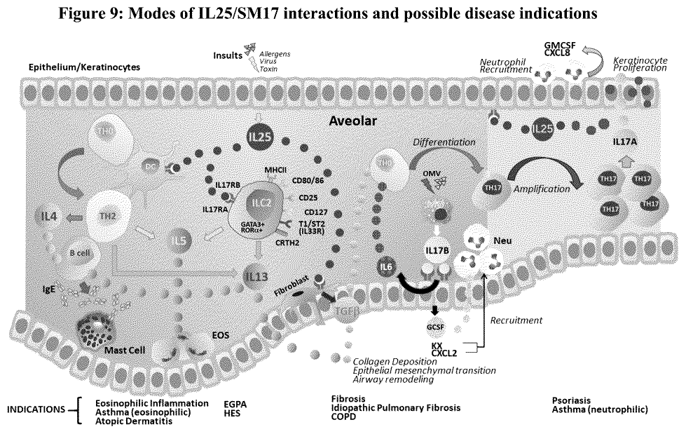

In addition, SM17 administered at 1, 3 and 5 mg/kg, significantly reduced the IL-5 levels in BALF. SM17 administered at 5 mg/kg significantly reduced the IL-13 levels and eosinophil number in BALF (FIG. 7). SM17 at 5 mg/kg and Dex was found to significantly reduce the number of pulmonary infiltrated eosinophils. Results are presented as means±SEM. One Way ANOVA, Dunnett's Multiple Comparison Test, *: P<0.05; **: P<0.01. Dex=Dexamethasone. FIG. 9 shows the proposed mechanism of action of SM17 to treat various indications.

### Example 8: Construction and Expression of Anti-Alarmin Bispecific Binding Proteins (bsBp)

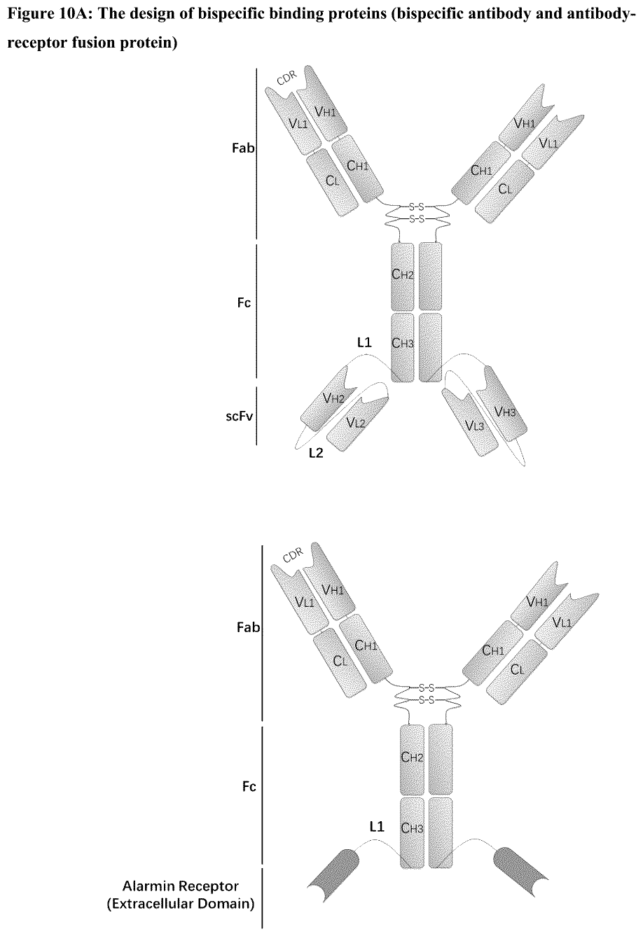

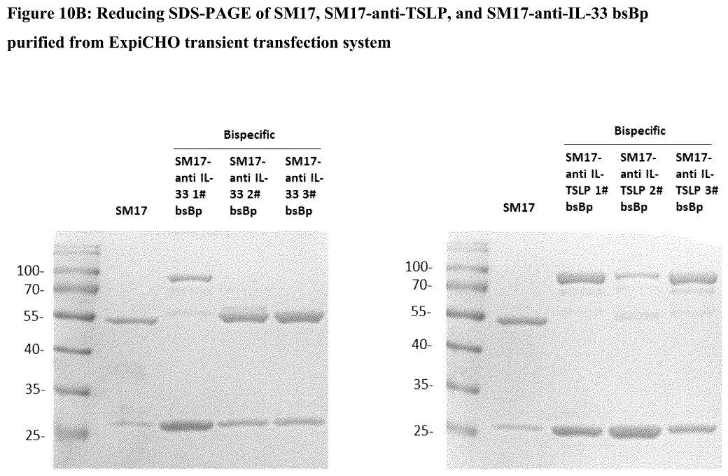

The light chain (SEQ ID NO: 33) of SM17 are cloned into pcDNA3.3 expression vector through TA cloning (Thermo Fisher Scientific). The cDNA coding the SM17 heavy chains that are operationally linked to the sequence of a particular alarmin binding protein (e.g., alarmin binding receptor or scFv of an alarmin specific antibody) are Gene-synthesized (Genscript Biotech Corp., Piscataway, NJ) and cloned into the NheI/NotI cloning site of the pEGFP-N1 expression vector (Clontech Laboratories, Mountain View, CA) (FIG. 10a). Expression vectors containing a particular SM17-alarmin binding protein heavy chain and expression vector for SM17 light chain are co-transfected into expiCHO-S cells according to manufacturer's specifications (Thermo Fisher Scientific). The particular bispecific binding proteins (bsBp) containing SM17 antibody linked to a particular alarmin binding moiety are harvested on day 12 post transfection and purified by protein A affinity chromotography. Reducing SDS-PAGE of purified bsBp is shown in FIG. 10b, demonstrating that most of the bispecific antibodies are intact in nature The heavy chain and light chain sequence of bsBp are summarized in Table 4 below.

TABLE 4
Amino acid sequences of bsBp and monoclonal antibody

| | HC | LC |
|---|---|---|
| | sequence | sequence |
| Construct | ID | ID |
| SM17-anti TSLP 1# bsBp | 60 | 33 |
| SM17-anti TSLP 2# bsBp | 61 | 33 |
| SM17-anti TSLP 3# bsBp | 62 | 33 |
| SM17-anti IL-33 1# bsBp | 63 | 33 |
| SM17-anti IL-33 2# bsBp | 64 | 33 |
| SM17-anti IL-33 3# bsBp | 65 | 33 |
| Anti-TSLP antibody 1# | 75 | 76 |
| Anti-TSLP antibody 2# | 77 | 78 |
| Anti-TSLP antibody 3# | 79 | 80 |
| Anti-IL-33 antibody 1# | 81 | 82 |
| Anti-IL-33 antibody 2# | 83 | 84 |
| Anti-IL-33 antibody 3# | 85 | 86 |
| SM17-hTSLPR | 67 | 33 |
| SM17-hST2 | 66, 74 | 33 |

### Example 9: Specificities of Anti-Alarmin bsBp

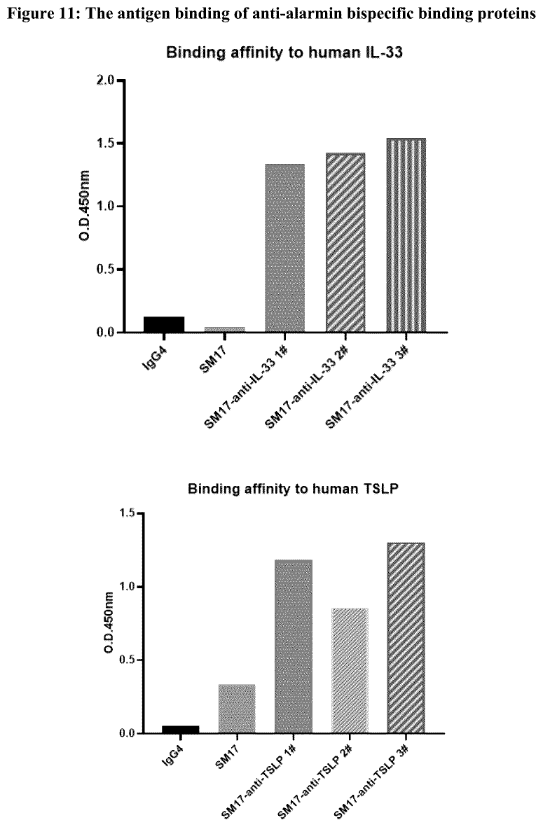

Binding specificities of bsBp were evaluated by standard ELISA assays. Briefly, ELISA strips were coated with the respective target antigens, including IL17RB, IL-33, or TSLP (R&D systems) at a final concentration of 2 μg/mL. Bispecific antibodies were added at 33.5 nM to ELISA strips coated with the desired antigen. After incubation under room temperature for 2 hours, ELISA strips were washed five times with PBS. Binding was revealed by the addition of goat anti-human F(ab′)2 specific horseradish peroxidase (HRP)-conjugated secondary antibodies (1:5000 dilution, Jackson ImmunoResearch) followed by TMB substrate solutions (Sigma-Aldrich-) according to standard procedures (FIG. 11). Antigen binding specificity of bsBp are summarized in FIG. 11 and Table 5 below. Results indicate specificities against IL17RB and the designed alarmins as originally designed. No result is provided for SM17-human TSLPR as the yield was too low to be detected.

TABLE 5
Antigen binding specificity of bsBp in ELISA

| Antigen | SM17- | SM17- | SM17- | SM17- | SM17- | SM17- | | |
|---|---|---|---|---|---|---|---|---|
| coated on | anti- | anti- | anti- | anti- | anti- | anti- | SM-17- | |
| ELISA | TSLP | TSLP | TSLP | IL-33 | IL-33 | IL-33 | human | |
| strips | 1# | 2# | 3# | 1# | 2# | 3# | ST2 | SM17 |
| TSLP | +++ | ++ | +++ | N/A | N/A | N/A | − | + |
| IL-17RB | +++ | +++ | +++ | +++ | +++ | +++ | +++ | +++ |
| IL-33 | N/A | N/A | N/A | +++ | +++ | +++ | +++ | − |

Intensity of specific antigen binding affinity on a semiquantitative scale of +/+++ for bsBp (− = no binding, +, ++, +++ = weak, moderate, strong binding), N/A refers to no binding affinity.

### Example 10: The Alarmin Binding Kinetics of Bispecific Binding Proteins

The binding affinity of bsBp to the targeted alarmins was determined using a bio-Layer Interferometry analysis (Octet ReD96 system, Sartorius). bsBp or SM17 (20 μg/mL) was immobilized on biosensors via binding to anti-human Fab CH1 region; serially diluted human IL33 and TSLP (158.7 nM, 79.4 nM, 39.7 nM) (R&D systems) were added to plot the association and dissociation curve according to the manufacturer's specification. Biosensors immobilized with irrelevant antibody (SM03, anti-human CD22 chimeric IgG1 antibody, SinoMab BioScience Limited, 20 μg/mL) were used as control reference. Expected Ka, Kdis and K_{D} values of the respective bispecific binding proteins are summarized in Table 6 below.

TABLE 6
Binding Kinetics of bsBp to human TSLP and IL-33

| | Kₐ | K_{dis} | K_{D} |
|---|---|---|---|
| TSLP | (1/Ms) | (1/s) | (nM) |
| SM17-anti-TSLP | 4.72 × 10⁵ | 2.41 × 10⁻⁴ | 0.511 |
| 1# bsBp | | | |
| SM17-anti-TSLP | 3.74 × 10⁵ | <1 × 10⁻⁷ | <0.001 |
| 2# bsBp | | | |
| SM17-anti-TSLP | 4.81 × 10⁵ | 3.17 × 10⁻⁵ | 0.0658 |
| 3# bsBp | | | |
| SM17-TSLPR | N/A | N/A | N/A |
| SM17 | 1 × 10⁴ | 1 × 10⁻³ | 100 |
| | Kₐ | K_{dis} | K_{D} |
| IL33 | (1/Ms) | (1/s) | (nM) |
| SM17-anti-IL-33 | 9.81 × 10³ | 2.83 × 10⁻⁴ | 28.9 |
| 1# bsBp | | | |
| SM17-anti-IL-33 | 3.73 × 10⁴ | <1 × 10⁻⁷ | 0.0259 |
| 2# bsBp | | | |
| SM17-anti-IL-33 | 2.27 × 10⁴ | <1 × 10⁻⁷ | 0.0425 |
| 3# bsBp | | | |
| SM17-ST2 | 5.56 × 10⁵ | 2.26 × 10⁻⁴ | 0.407 |
| SM17 | 2.72 × 10⁴ | 1.79 × 10¹ | 659000 |

No binding affinity is given to SM17-human TSLPR as the yield is too low.

### Example 11: Induction of IFNγ, CCL8, CCL17, IL-5 and IL-13 Releases from Human PBMC by Alarmins and the Inhibitory Effects of SM17 on Cytokine Release

Human PBMCs (4×10⁵ per group, Ixcells) were incubated with IL-2 (10 unit/mL, PeproTech) and various alarmin/alarmin combinations to mimic the pro-inflammatory cytokines release during allergic diseases. For IL-5 and IL-13 releases, PBMCs were incubated with IL-2 and three alarmins (all 10 ng/ml) for three days. For CCL17 release, PBMC was incubated with TSLP (10 ng/ml) for 1 day. For CCL8 and IFNγ releases, PBMCs were incubated with IL-12 (10 ng/ml, Sino Biological) and IL-33 (10 ng/ml) for 1 day. Cells were pretreated with SM17 (5 μg/ml) or IgG4 isotype control (5 μg/ml) for an hour before the addition of cytokines.

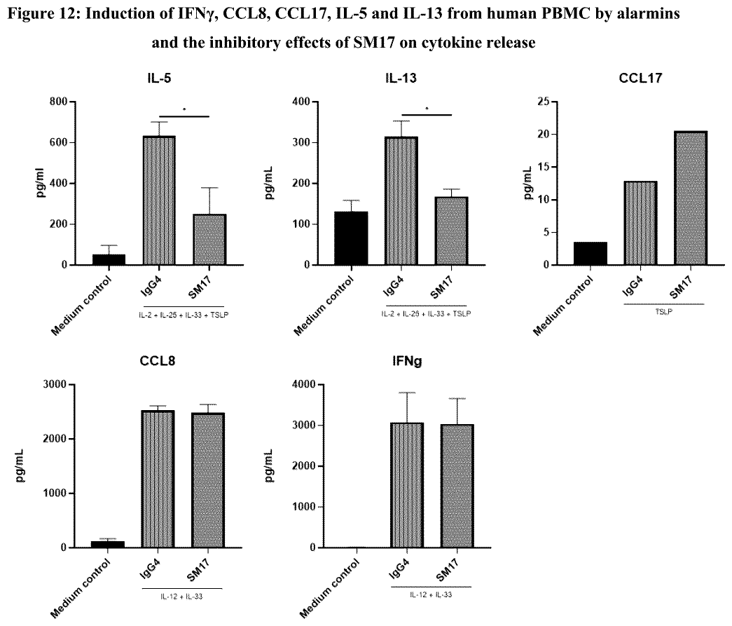

Supernatants were collected and the cytokine concentration were measured by ELISA kits (R&D systems). These established assays were used to test the potency of and SM17 and bsBps. SM17 could effectively suppress the secretion of IL-5 and IL-13 in total alarmin stimulated PBMC cultures. However, no inhibitory effects of SM17 treatments were indicated in CCL17, CCL8 and IFNγ release assays as compared to IgG4 isotype control (See FIG. 12). Results were expressed in absolute amount in pg/ml.

### Example 12: Potency of Different Bispecific Binding Proteins on Suppressing Cytokine and Chemotactic Factor Releases from Induced Human PBMC

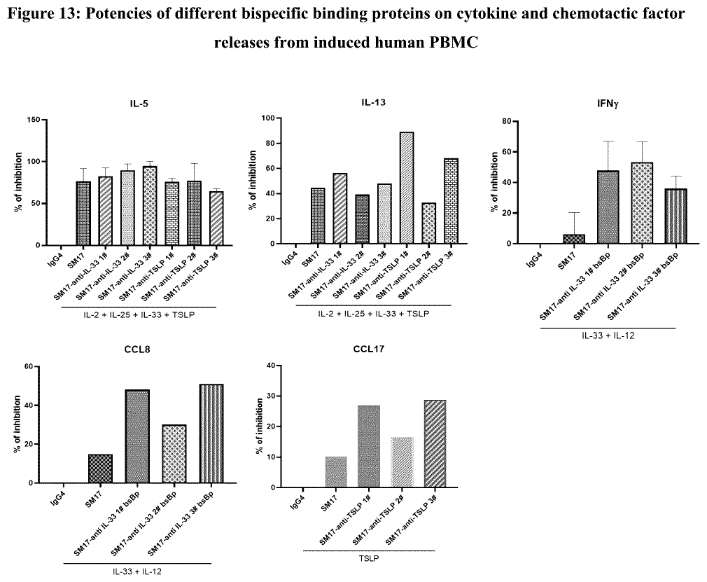

SM17, three different SM17-anti-IL33 bsBps and three different SM17-anti-TSLP bsBps (all at 5 μg/ml) were added to human PBMC cultures under conditions that shall induce the release of IFNγ, CCL8, CCL17 and IL-5 as described in Example 11 above. The levels of different cytokines and chemotactic factors in supernatant obtained in induced PBMC were evaluated using standard ELISA methods. Results indicate that different pairs of SM17/anti-TSLP and SM17/anti-IL33 bsBps exhibit enhanced or/and synergistic effects in suppressing the releases of IFNγ, CCL8, CCL17, IL-5 and IL-13 when compared to that observed with SM17 alone (See FIG. 13). Specifically, in IL-5 release assay, SM17-anti-IL-33 3 # showed the strongest suppression effect as compared to SM17 treatment. In the CCL8 release assay, both SM17-anti-IL-33 1 # and SM17-anti-IL-33 3 # showed the stronger suppression effect as compared to SM17. In IFNγ release assay, all three SM17-anti-IL-33 bsBps showed stronger suppression effect as compared to SM17. In CCL17 release assay, both SM17-anti-TSLP 1 # and SM17-anti-TSLP 3 #bsBps showed stronger suppression effect as compared to SM17 treatment. The potencies of bsBp in different cytokine release assays are summarized in FIG. 13 and Table 7.

TABLE 7
Potencies of bsBp in cytokine release assay

| | SM17- | SM17- | SM17- | SM17- | SM17- | SM17- | |
|---|---|---|---|---|---|---|---|
| | anti TSLP | anti TSLP | anti TSLP | anti IL-33 | anti IL-33 | anti IL-33 | |
| Cytokines | 1# bsBp | 2# bsBp | 3# bsBp | 1# bsBp | 2# bsBp | 3# bsBp | SM17 |
| IL-5 | ++ | ++ | + | ++ | ++ | +++ | ++ |
| IL-13 | +++ | ++ | +++ | ++ | ++ | ++ | ++ |
| CCL-8 | Nil | Nil | Nil | + | + | ++ | − |
| CCL-17 | + | + | + | Nil | Nil | Nil | − |
| IFNγ | Nil | Nil | Nil | ++ | ++ | + | − |

Intensity of specific cytokine inhibition on a semiquantitative scale of +/+++ for bsBp (− = no inhibition, +, ++, +++ = weak, moderate, strong inhibition).
Nil: Not investigated.

### Example 13: Pro-Proliferative Effects of Alarmins on ILC2

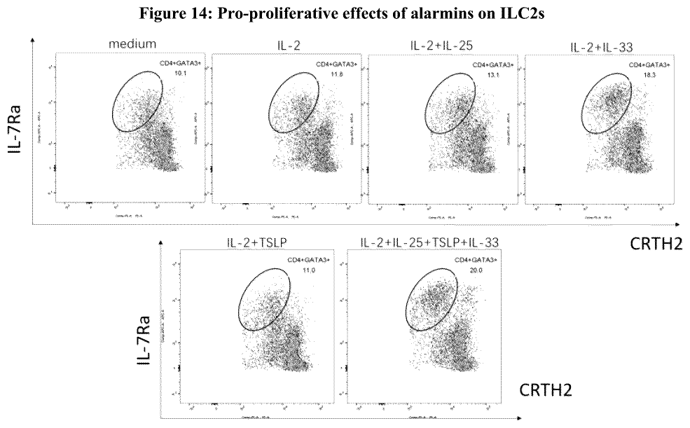

Isolated human ILC2 cells can proliferate in response to alarmins. Human ILC2 was enriched from fresh human PBMC by EasySep™ Human ILC2 Isolation Kit (STEMCELL Technologies Inc. Cambridge, MA). Concentrated ILC2 was then cultured in RPMI1640 medium (Thermo Fisher Scientific) with 10% human AB serum (Sigma). IL-2 (10 unit/mL) together with IL-25 (10 ng/mL) or TSLP (10 ng/mL) or IL-33 (10 ng/mL) or a combination of these 3 alarmins are added into ILC2 cultures for 7 days before the changes in ILC2 cell population were measured by flow cytometry (BD FACSVerse) using a panel of lineage-specific antibodies (CRTH2+, IL-7Ra+; BioLegend, San Diego, CA) against ILC2. Results indicate that the main contributor for ILC2 proliferation is IL-33 and the combination of all alarmins further potentiate ILC2 expansion (See FIG. 14).

### Example 14: Suppression of ILC2 and Th2 Cell Population in Human PBMC by Dexamethasone and bsBp

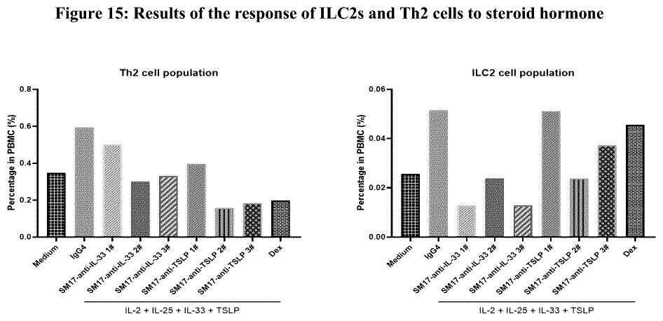

While alarmins are known to work in concert to enhance ILC2 and Th2 cell functions such as increase in cell population, steroids such as Dexamethasone (Dex) can negate these effects, resulting in the mitigation of type 2 immune responses [Jia, Yi et al. American Journal Of Respiratory Cell And Molecular Biology, vol. 55, 5 (2016): 675-683]. ILC2 and Th2 cell population can be determined by flow cytometry (BD FACSVerse) following standard procedures known to those skilled in the art. Herein, the cell population of ILC2 and Th2 cells was experimentally enhanced by a combination of IL-2 (10 unit/mL) and three alarmins (10 ng/ml) for 5 days. Results demonstrate that Dex can significantly reduce the Th2 cell population (CD4+GATA3+ population, gated by anti-CD4 PE antibody (BioLegend) and anti-GATA3 APC antibody (Abcam, Cambridge, UK) but not ILC2 population (lin−IL-7Ra+CRTH2+, gated by anti-IL-7Ra percp/cyanine5.5 antibody and anti-CRTH2 FITC antibody, BioLegend) in human PBMC culture (see FIG. 15). Furthermore, addition of Bsbp significantly reduces ILC2 cell population and the potency of bsBp are summarized in FIG. 15 and Table 8 below.

TABLE 8
Potencies of bsBp in ILC2 and Th2 proliferation assay

| | SM17-anti | SM17-anti | SM17-anti | SM17-anti | SM17-anti | SM17-anti | | |
|---|---|---|---|---|---|---|---|---|
| Cell | TSLP 1# | TSLP 2# | TSLP 3# | IL-33 1# | IL-33 2# | IL-33 3# | | |
| type | bsBp | bsBp | bsBp | bsBp | bsBp | bsBp | SM17 | Dex |
| ILC2 | + | +++ | ++ | +++ | +++ | +++ | ++ | + |
| Th2 | ++ | +++ | +++ | + | +++ | +++ | +++ | +++ |

Cell proliferation inhibition on a semiquantitative scale of +/+++++ for bsBp (− = no inhibition, +, ++, +++ = weak, moderate, strong inhibition)

### Example 15: Dendritic Cell Potency Assay

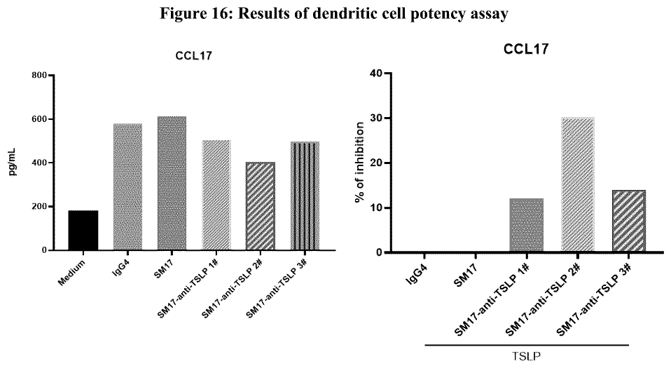

TSLP from inflamed epithelium promotes maturation of dendritic cells (DCs) to prime Th2 responses via CCL17, which induces chemotaxis of CD4+ T cells to mediate inflammation (Kitajima and Ziegler. 2013. “Cutting Edge: Identification Of The Thymic Stromal Lymphopoietin-Responsive Dendritic Cell Subset Critical For Initiation Of Type 2 Contact Hypersensitivity”. J Immunol 191: 4903-4907; Bleck et al. 2015. “Co-Expression Of Type 2 Immune Targets In Sputum-Derived Epithelial And Dendritic Cells From Asthmatic Subjects”. J Allergy Clin Immunol 136: 619-627.e5). Normal human DCs were obtained from Lonza (Bend, OR). The DCs were cultured in LGM-3 medium (Lonza). TSLP (10 ng/mL) is added into the DC cultures for 5 days in the presence of 5 μg/mL of SM17, IgG4 isotype control (Sino Biological), SM17-TSLPR bsBp (SM17 fused with TSLP receptor extracellular domain) or SM17-anti-TSLP bsBp 2 #. The supernatants are harvested and the levels of CCL17 measured by ELISA following the manufacturer's specification (R&D systems). Results indicate that bispecific SM17-TSLPR and SM17-anti-TSLP bsBp 2 # is efficient in suppressing TSLP-induced CCL17 production in DCs (FIG. 16). The potencies of SM17, IgG4, and bsBp are summarized in Table 9 below.

TABLE 9
Potencies of bsBp in CCL17 release assay on dendritic cells

| | SM17- | SM17- | SM17- | | |
|---|---|---|---|---|---|
| | anti | anti | anti | | |
| | TSLP 1# | TSLP 2# | TSLP 3# | | |
| Construct | bsBp | bsBp | bsBp | SM17 | IgG4 |
| Potency | − | + | − | − | − |

The inhibition of CCL17 release on a semiquantitative scale of +/+++ for bsBp, SM17, and IgG4 isotype (− = no inhibition, +, ++, ++ = weak, moderate, strong inhibition)

While the invention has been described in detail and with reference to specific embodiments thereof, it will be apparent to one skilled in the art that various changes and modifications can be made therein without departing from the spirit and scope of the invention.

What is claimed:

1. A method of treating a disease or disorder related to allergy in a subject in need thereof, said method comprising the step of delivering to cells or tissues of the subject a therapeutically effective amount of a bispecific binding protein against 2 different alarmins X and Y, wherein said bispecific binding protein consists of (a) an anti-alarmin X receptor IgG and (b) an anti-alarmin Y scFv and (c) a polypeptide linker.

2. A method of treating a disease or disorder related to allergy in a subject in need thereof, said method comprising the step of delivering to cells or tissues of the subject a therapeutically effective amount of a bispecific binding protein against 2 different alarmins X and Y, wherein said bispecific binding protein consists of (a) an anti-alarmin X receptor IgG and (b) an alarmin Y receptor and (c) polypeptide linker.

3. The method of claim 1, wherein the alarmin X receptor is IL-17RB.

4. The method of claim 1, wherein the alarmin Y is TSLP or IL-33.

5. The method of claim 2, wherein the alarmin Y receptor is ST2 or TSLPR.

6. The method of claim 1, wherein the anti-alarmin X receptor IgG is an antibody selected from the group consisting of an IgG1 antibody, an IgG2 antibody, an IgG3 antibody, and an IgG4 antibody.

7. The method of claim 1, wherein the subject has clinical or pre-clinical asthma, atopic dermatitis, fibrotic disease, inflammatory bowel disease (IBD), Crohn's disease, ulcerative colitis, chronic obstructive pulmonary disease, chronic sinusitis, chronic rhinosinusitis with nasal polyps.

8. A method of treating a disease or disorder related to allergy in a subject in need thereof, said method comprising the step of delivering to cells or tissues of the subject a therapeutically effective amount of a bispecific binding protein that blocks (a) IL-25 and IL-33 signaling and/or (b) IL-25 and TSLP signaling.

9. The method of claim 8, wherein the subject has clinical or pre-clinical asthma, atopic dermatitis, fibrotic disease, inflammatory bowel disease (IBD), Crohn's disease, ulcerative colitis, chronic obstructive pulmonary disease, chronic sinusitis, chronic rhinosinusitis with nasal polyps.

10. The method of claim 8, wherein bispecific binding protein is an anti-IL-17RB/anti-human TSLP bispecific antibody.

11. The method of claim 8, wherein bispecific binding protein is an anti-IL-17RB/anti-human IL-33 bispecific antibody.

12. The method of claim 8, wherein bispecific binding protein is an anti-IL-17RB/human ST2 antibody-receptor fusion protein.

13. The method of claim 8, wherein bispecific binding protein is an anti-IL-17RB/human TSLPR antibody-receptor fusion protein.

14. The method of claim 8, wherein the bispecific binding protein comprises a light chain variable region (VL) comprising a VL CDR1, VL CDR2, and VL CDR3 that have the amino acid sequences of SEQ ID NO: 4, SEQ ID NO: 5, and SEQ ID NO: 6, respectively; and a heavy chain variable region (VH) comprising a VH CDR1, VH CDR2, and VH CDR3 that have the amino acid sequence of SEQ ID NO:1, SEQ ID NO:2, and SEQ ID NO:3, respectively.

15. The method of claim 14, wherein the VL and VH of the anti-IL-17RB antibody have the amino acid sequences of SEQ ID NO:33 and SEQ ID NO:32, respectively.

16. The method of claim 8, wherein the bispecific binding protein comprises a light chain sequence of SEQ ID NO: 33 and a heavy chain sequence selected from the group consisting of SEQ ID NOs: 60-74.

17. The method of claim 1, wherein the bispecific binding protein is delivered to said cells or tissues intravenously, intramuscularly, subcutaneously, intracranially, intrathecally, intraventricularly, intraperitoneally, intranasally, parenterally, topically, or intradermally.

18. The method of claim 1, wherein bispecific binding protein is delivered in combination with a second therapeutic agent.

19. The method of claim 18, wherein the second therapeutic agent is selected from the group consisting of a corticosteroid, a DNA methyltransferase (DNMT) inhibitor, an anti-IL17A antibody, an anti-IL12/IL23 antibody, an anti-IL23 antibody, an anti-IL17RA antibody, and a tyrosine kinase inhibitor.

20. The method of claim 1, wherein the subject is a human subject and the cells or tissues of the subject are immune cells obtained and isolated from said subject.


SEQUENCE LISTING (87 sequences)

SEQ ID NO: 1 - protein, 5 aa, synthetic construct
SYWMN

SEQ ID NO: 2 - protein, 17 aa, synthetic construct
RIDPYDSEIQYNQKFKD

SEQ ID NO: 3 - protein, 9 aa, synthetic construct
SGGFDWFAY

SEQ ID NO: 4 - protein, 11 aa, synthetic construct
RASENINSNLA

SEQ ID NO: 5 - protein, 7 aa, synthetic construct
DVTNLAD

SEQ ID NO: 6 - protein, 9 aa, synthetic construct
QHFWGPPYT

SEQ ID NO: 7 - protein, 5 aa, synthetic construct
DYAMH

SEQ ID NO: 8 - protein, 17 aa, synthetic construct
TFIPLLDTSDYAQKFQG

SEQ ID NO: 9 - protein, 11 aa, synthetic construct
MGVTHSYVMDA

SEQ ID NO: 10 - protein, 11 aa, synthetic construct
RASQPISISVH

SEQ ID NO: 11 - protein, 7 aa, synthetic construct
FASQSIS

SEQ ID NO: 12 - protein, 9 aa, synthetic construct
QQTFSLPYT

SEQ ID NO: 13 - protein, 5 aa, synthetic construct
RSAMN

SEQ ID NO: 14 - protein, 17 aa, synthetic construct
GISGSGGRTYYADSVKG

SEQ ID NO: 15 - protein, 13 aa, synthetic construct
DSYTTSWYGGMDV

SEQ ID NO: 16 - protein, 11 aa, synthetic construct
RASQGIFSWLA

SEQ ID NO: 17 - protein, 7 aa, synthetic construct
AASSLQS

SEQ ID NO: 18 - protein, 9 aa, synthetic construct
QQANSVPIT

SEQ ID NO: 19 - protein, 209 aa, synthetic construct
QGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 20 - protein, 310 aa, synthetic construct
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 21 - protein, 18 aa, synthetic construct
VDGASSPVNVSSPSVQDI

SEQ ID NO: 22 - protein, 18 aa, synthetic construct
VDGASSPVNVGSPSVQDI

SEQ ID NO: 23 - protein, 13 aa, synthetic construct
GASSPVNVSSPSV

SEQ ID NO: 24 - protein, 18 aa, synthetic construct
GGGGSGGGNGTGSGGGGS

SEQ ID NO: 25 - protein, 18 aa, synthetic construct
LSAGGHGGLDNDTSAFHL

SEQ ID NO: 26 - protein, 18 aa, synthetic construct
SDKTHTSPPSPAPESSGG

SEQ ID NO: 27 - protein, 18 aa, synthetic construct
VTTTDFQIQTEMAATMET

SEQ ID NO: 28 - protein, 18 aa, synthetic construct
DFLPTTAQPTKKSTLKKR

SEQ ID NO: 29 - protein, 18 aa, synthetic construct
TESRSPPAENEVSTPMQA

SEQ ID NO: 30 - protein, 18 aa, synthetic construct
GGGGSGGGNGSGSGGGGS

SEQ ID NO: 31 - protein, 18 aa, synthetic construct
GGGGSGGGSGGGSGGGGS

SEQ ID NO: 32 - protein, 443 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 33 - protein, 214 aa, synthetic construct
DIQMTQSPSSLSASVGDRVTITCRASENINSNLAWYQQKPGKAPKLLLYDVTNLADGVPSRFSGSGSGTDYTLTISSLQPEDFATYYCQHFWGPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 213 aa, synthetic construct
AAAVTSRGDVTVVCHDLETVEVTWGSGPDHHGANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPARCYDFRVRASPRAAHYGLEAQPSEWTAVTRLSGAASAASCTASPAPSPALAPP

SEQ ID NO: 35 - protein, 306 aa, synthetic construct
SKSSWGLENEALIVRCPQRGRSTYPVEWYYSDTNESIPTQKRNRIFVSRDRLKFLPARVEDSGIYACVIRSPNLNKTGYLNVTIHKKPPSCNIPDYLMYSTVRGSDKNFKITCPTIDLYNWTAPVQWFKNCKALQEPRFRAHRSYLFIDNVTHDDEGDYTCQFTHAENGTNYIVTATRSFTVEEKGFSMFPVITNPPYNHTMEVEIGKPASIACSACFGKGSHFLADVLWQINKTVVGNFGEARIQEEEGRNESSSNDMDCLTSVLRITGVTEKDLSLEYDCLALNLHGMIRHTIRLRRKQPIDHR

SEQ ID NO: 36 - protein, 5 aa, synthetic construct
TYGMH

SEQ ID NO: 37 - protein, 17 aa, synthetic construct
VIWYDGSNKHYADSVKG

SEQ ID NO: 38 - protein, 13 aa, synthetic construct
APQWELVHEAFDI

SEQ ID NO: 39 - protein, 11 aa, synthetic construct
GGNNLGSKSVH

SEQ ID NO: 40 - protein, 7 aa, synthetic construct
DDSDRPS

SEQ ID NO: 41 - protein, 11 aa, synthetic construct
QVWDSSSDHVV

SEQ ID NO: 42 - protein, 5 aa, synthetic construct
SYYMS

SEQ ID NO: 43 - protein, 16 aa, synthetic construct
GIFFDGETYYAGSVKG

SEQ ID NO: 44 - protein, 9 aa, synthetic construct
QQYFDHIDI

SEQ ID NO: 45 - protein, 11 aa, synthetic construct
GGDSLGGKYVY

SEQ ID NO: 46 - protein, 7 aa, synthetic construct
GDSKRPS

SEQ ID NO: 47 - protein, 11 aa, synthetic construct
QSYDLQSLNIV

SEQ ID NO: 48 - protein, 5 aa, synthetic construct
DYYMN

SEQ ID NO: 49 - protein, 17 aa, synthetic construct
SISRYSSYIYYADSVKG

SEQ ID NO: 50 - protein, 7 aa, synthetic construct
DIGGMDV

SEQ ID NO: 51 - protein, 14 aa, synthetic construct
TGSSSNIGAVYDVH

SEQ ID NO: 52 - protein, 7 aa, synthetic construct
RNNQRPS

SEQ ID NO: 53 - protein, 9 aa, synthetic construct
QTYDSSRWV

SEQ ID NO: 54 - protein, 5 aa, synthetic construct
SYWMH

SEQ ID NO: 55 - protein, 17 aa, synthetic construct
TIYPRNSNTDYNQKFKA

SEQ ID NO: 56 - protein, 13 aa, synthetic construct
PLYYYLTSPPTLF

SEQ ID NO: 57 - protein, 11 aa, synthetic construct
KASQDVGTAVA

SEQ ID NO: 58 - protein, 7 aa, synthetic construct
WASTRHT

SEQ ID NO: 59 - protein, 9 aa, synthetic construct
QQAKTYPFT

SEQ ID NO: 60 - protein, 734 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSGGGGSGGGGSGGGGSGGGGSSYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 61 - protein, 731 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSGGGGSGGGGSGGGGSGGGGSEIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIK

SEQ ID NO: 62 - protein, 731 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQVQLVESGGGLVQPGGSLRLSCAASGFTFSSYYMSWVRQAPGKGLEWVSGIFFDGETYYAGSVKGRFTISRDNSKNTLYLQMNSLRAEDTAVYYCARQQYFDHIDIWGQGTLVTVSSGGGGSGGGGSGGGGSGGGGSDIELTQPPSVSVAPGQTARISCGGDSLGGKYVYWYQQKPGQAPVLVIYGDSKRPSGIPERFSGSNSGNTATLTISGTQAEDEADYYCQSYDLQSLNIVFGGGTKLTVLGQ

SEQ ID NO: 63 - protein, 733 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSSGGGGSGGGGSGGGGSGGGGSDIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIK

SEQ ID NO: 64 - protein, 733 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSEVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSGGGGSGGGGSGGGGSGGGGSDIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 65 - protein, 729 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSEVQLLESGGGLVQPGGSLRLSCAASGSTFSDYYMNWVRQAPGKGLEWVSSISRYSSYIYYADSVKGRFTISRDNSKNTLYLQMNSLRAEDTAVYYCARDIGGMDVWGQGTLVTVSSGGGGSGGGGSGGGGSGGGGSQSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 66 - protein, 794 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 67 - protein, 693 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 68 - protein, 713 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSGGGGSGGGGSGGGGSGGGGSSYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 69 - protein, 710 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSGGGGSGGGGSGGGGSGGGGSEIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIK

SEQ ID NO: 70 - protein, 710 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGFTFSSYYMSWVRQAPGKGLEWVSGIFFDGETYYAGSVKGRFTISRDNSKNTLYLQMNSLRAEDTAVYYCARQQYFDHIDIWGQGTLVTVSSGGGGSGGGGSGGGGSGGGGSDIELTQPPSVSVAPGQTARISCGGDSLGGKYVYWYQQKPGQAPVLVIYGDSKRPSGIPERFSGSNSGNTATLTISGTQAEDEADYYCQSYDLQSLNIVFGGGTKLTVLGQ

SEQ ID NO: 71 - protein, 712 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSSGGGGSGGGGSGGGGSGGGGSDIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIK

SEQ ID NO: 72 - protein, 712 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSGGGGSGGGGSGGGGSGGGGSDIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 73 - protein, 708 aa, synthetic construct
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGSTFSDYYMNWVRQAPGKGLEWVSSISRYSSYIYYADSVKGRFTISRDNSKNTLYLQMNSLRAEDTAVYYCARDIGGMDVWGQGTLVTVSSGGGGSGGGGSGGGGSGGGGSQSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 74 - protein, 794 aa, synthetic construct
MDLLHKNMKHLWFFLLLVAAPRWVLSQVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHS

SEQ ID NO: 75 - protein, 452 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 215 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 450 aa, synthetic construct
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 447 aa, synthetic construct
QVQLVESGGGLVQPGGSLRLSCAASGFTFSSYYMSWVRQAPGKGLEWVSGIFFDGETYYAGSVKGRFTISRDNSKNTLYLQMNSLRAEDTAVYYCARQQYFDHIDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 217 aa, synthetic construct
DIELTQPPSVSVAPGQTARISCGGDSLGGKYVYWYQQKPGQAPVLVIYGDSKRPSGIPERFSGSNSGNTATLTISGTQAEDEADYYCQSYDLQSLNIVFGGGTKLTVLGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 452 aa, synthetic construct
QVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 214 aa, synthetic construct
DIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 452 aa, synthetic construct
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, synthetic construct
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 446 aa, synthetic construct
EVQLLESGGGLVQPGGSLRLSCAASGSTFSDYYMNWVRQAPGKGLEWVSSISRYSSYIYYADSVKGRFTISRDNSKNTLYLQMNSLRAEDTAVYYCARDIGGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 216 aa, synthetic construct
QSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 502 aa, synthetic construct
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL